(19) United States
(12) Patent Application Publication
(10) Pub. No.: US 2006/0134630 A1
(43) Pub. Date: Jun. 22, 2006

(54) Method for screening for an antimicrobial polypeptide

(75) Inventors: Dorotea Raventos Segura, Humlebaek (DK); Anja Lykke Hansen, Copenhagen V (DK); Per Holse Mygind, Soborg (DK); Hans-Henrik Kristensen Hogenhaug, Holte (DK); Ida Ellingsgaard, Lyngby (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 10/682,104

(22) Filed: Oct. 9, 2003

Related U.S. Application Data
(60) Provisional application No. 60/419,050, filed on Oct. 16, 2002.

(30) Foreign Application Priority Data
Oct. 9, 2002 (DK) PA 2002 01518
Dec. 2, 2002 (DK) PA 2002 01854

(51) Int. Cl. C12Q 1/68; C40B 40/08

(52) U.S. Cl. 435/6

(57) ABSTRACT

The present invention relates to a method for screening polynucleotide sequences encoding antimicrobial polypeptides and methods for testing the antimicrobial activity of an antimicrobial polypeptide.

### CROSS-REFERENCE TO RELATED APPLICATIONS

This application claims, under 35 U.S.C. 119, the benefit of Danish application no. PA 2002 01518, filed Oct. 9, 2002, and PA 2002 01854 filed on Dec. 2, 2002 and U.S. provisional application no. 60/419,050, filed Oct. 16, 2002, the contents of which are fully incorporated herein by reference.

### FIELD OF THE INVENTION

The present invention relates to a method for screening a polynucleotide sequence encoding an antimicrobial polypeptide for its antimicrobial activity.

### BACKGROUND OF THE INVENTION

Various bioactive polypeptides are known to kill or inhibit the proliferation of target cells, e.g. antimicrobial enzymes, anti-tumor peptides and antimicrobial polypeptides. Bioactive polypeptides have a variety of applications for example antimicrobial polypeptides may be used for various medical applications, such as to combat infections. As it is often difficult to both express and test the activity of an antimicrobial polypeptide because its activity may often interfere with expression in a host cell new methods for testing the activity of such polypeptides are desirable.

WO 00/73433 discloses a method for screening nucleotide sequences encoding anti microbial peptides comprising a) ligating a plasmid with the pool of nucleotide sequences linked to an inducible promoter, b) transforming host cells which are sensitive to the peptide with the ligated plasmids, c) screening the transformed host cells so as to select viable cells, d) cultivating the viable cells in the presence of inducer so as to induce expression of said nucleotide sequence, e) selecting cells according to the effect of the inducer on cell growth. Reichhart J-M et al. (1992), Invertebrate Reproduction and Development, 21 (1), pp. 15-24 discloses expression and secretion in yeast of active insect defensin, an inducible antibacterial peptide from the fleshfly Phormia terranovae.

Teilum K et al. (1999), Protein Expression and Purification 15, pp. 77-82 discloses that the yield of ATP N peroxidase can be increased by using thioredoxin reductase negative strains, which facilitate the formation of disulfide bonds in inclusion body protein.

Kekessy D A and Piguet J D, Applied Microbiology, 20 (2), pp. 282-283 discloses a new method for detecting Bacteriocin production.

### SUMMARY OF THE INVENTION

The present invention relates to a method for screening a polynucleotide sequence encoding an antimicrobial polypeptide, said method comprising the steps of:
a) introducing the polynucleotide sequence in a host cell, wherein expression of said polynucleotide is under control of an inducible promoter and wherein said host cell is sensitive to the antimicrobial peptide encoded by the polynucleotide sequence
b) cultivating the host cells of in the absence of an inducer capable of inducing expression of the antimicrobial peptide
c) cultivating the host cells in the presence of an inducer capable of inducing expression of the antimicrobial peptide
d) cultivating the host cells in the presence of an indicator cell, wherein said indicator cell is sensitive to the antimicrobial polypeptide encoded by the polynucleotide sequence
e) selecting host cells capable of reducing the proliferation of indicator cells
f) recovering the polynucleotide sequence encoding an antimicrobial polypeptide from the host cells selected in step e).

The present invention also relates to a method for screening a library of polynucleotide sequences encoding one or more antimicrobial polypeptide(s), comprising:
a) cultivating an indicator cell in the presence of the antimicrobial polypeptide(s), wherein the antimicrobial polypeptide(s) has/have been expressed by a host which is sensitive to said antimicrobial polypeptide(s), and wherein expression of each of the polynucleotide sequences in the library has been under control of an inducible promoter, and wherein the host cells comprising the library polynucleotide sequence has been cultivated in the absence of an inducer and subsequently in the presence of an inducer
b) selecting a host cell expressing an antimicrobial polypeptide which is capable of reducing the proliferation of the indicator cell

The present invention also relates to a method for testing the antimicrobial activity of an antimicrobial polypeptide comprising:
a) cultivating an indicator cell in the presence of the antimicrobial polypeptide(s), wherein the antimicrobial polypeptide(s) has/have been expressed by a host which is sensitive to said antimicrobial polypeptide(s), and wherein expression of each of the polynucleotide sequences in the library has been under control of an inducible promoter, and wherein the host cells comprising the library polynucleotide sequence has been cultivated in the absence of an inducer and subsequently in the presence of an inducer

Furthermore, the present invention relates to antimicrobial polypeptides identified by these methods.

### DEFINITIONS

The term “antimicrobial polypeptide” (AMP) is in the context of the present invention to be understood as a polypeptide having an antimicrobial activity.

The term “antimicrobial activity” is in the context of the present invention to be understood as an activity which is capable of killing or inhibiting the proliferation of microorganisms, including bacteria, viruses, unicellular algae and protozoans, and fungi. In the context of the present invention the term “antimicrobial” is intended to mean that there is a bactericidal and/or a bacteriostatic and/or fungicidal and/or fungistatic effect and/or a virucidal effect, wherein the term “bactericidal” is to be understood as capable of killing bacterial cells. The term “bacteriostatic” is to be understood as capable of inhibiting bacterial proliferation, i.e. inhibiting proliferating bacterial cells. The term “fungicidal” is to be understood as capable of killing fungal cells. The term “fungistatic” is to be understood as capable of inhibiting fungal proliferation, i.e. inhibiting proliferating fungal cells. The term “virucidal” is to be understood as capable of inactivating virus. The term “proliferation” or “proliferating” may be used interchangeably with the terms “growth” or “growing” in the present invention.

For purposes of the present invention, antimicrobial activity may be determined according to the procedure described by Lehrer et al., Journal of Immunological Methods, Vol. 137 (2) pp. 167-174 (1991).

Polypeptides having antimicrobial activity may be capable of reducing the number of living cells of Escherichia coli (DSM 1576) to 1/100 after 30 min. incubation at 20°C. in an aqueous solution of 25% (w/w); particularly in an aqueous solution of 10% (w/w); more particularly in an aqueous solution of 5% (w/w); even more particularly in an aqueous solution of 1% (w/w); most particularly in an aqueous solution of 0.5% (w/w); and in particular in an aqueous solution of 0.1% (w/w). Polypeptides having antimicrobial activity may also be capable of inhibiting the outgrowth of Escherichia coli (DSM1576) for 24 hours at 25° C. in a microbial growth substrate, when added in a concentration of 1000 ppm; particularly when added in a concentration of 500 ppm; more particularly when added in a concentration of 250 ppm; even more particularly when added in a concentration of 100 ppm; most particularly when added in a concentration of 50 ppm; and in particular when added in a concentration of 25 ppm.

The term “library” is in the context of the present invention to be understood as a collection of at least two different compounds, i.e. the term “library of polynucleotide sequences” means a collection of at least two different polynucleotide sequences. Within this context the term “different polynucleotide sequences” is to be understood as polynucleotide sequences which are different in regards to at least one nucleotide, e.g. the number of nucleotides in the sequences (i.e. the length of the sequence) or the identity of a nucleotide at a given position may be different. The term “library of indicator cells” refers to a collection of at least two different indicator cells, wherein the term “different indicator cells” is to be understood as cells which have a different genotype and/or different phenotype.

The term “polynucleotide” or “polynucleotide sequence” is in the context of the present invention to be understood as a chain of two or more nucleotides including, but not limited to, cDNA sequences, RNA sequences, genomic DNA sequences, synthetic or semi-synthetic nucleotide sequences or any combination thereof.

The term “polypeptide” refers in the context of the present invention to a peptide comprising two or more amino acids. Thus the term includes short chains of amino acids, such as between 2-100 amino acids and proteins with a defined three-dimensional structure.

The term “inducible promoter” is in the context of the present invention to be understood as a promoter from which transcription can be regulated, i.e. induced or repressed, by the presence or absence of a given compound. The term “regulator” is in this context to be understood as a compound which is capable of inducing or repressing transcription from an inducible promoter, e.g. the regulator may an “inducer”, i.e. a compound capable of inducing transcription, or it may be a “repressor”, i.e. a compound capable of repressing transcription from the inducible promoter.

The term “host cell” is in the context of the present invention to be understood as any cell which is susceptible to transformation, transfection or infection with a nucleic acid construct.

The term “nucleic acid construct” is in the context of the present invention to be understood as a polynucleotide sequence comprising a polynucleotide sequence encoding a polypeptide and the polynucleotide sequences necessary for expression of said polypeptide, e.g. said construct may be a plasmid, a bacteriophage or a virus.

The term “indicator cell” is in the context of the present invention to be understood as any cell for which it is of interest to screen a polynucleotide sequence encoding an antimicrobial polypeptide by the present invention. The term “target cell” is used interchangeably with “indicator cell” throughout this application.

The term “sensitive” or “sensitive to” used in relation to the sensitivity of an indicator or host cell towards an antimicrobial polypeptide is in the context of the present invention to be understood as the proliferation of said indicator/host cell is reduced or said indicator/host cell is killed by the presence, synthesis and/or expression of an AMP. Within the context of the present invention the indicator cells are typically affected by the presence of an AMP, while the host cells are typically affected by the synthesis and/or expression of an AMP.

The term “parent” is in the context of the present invention to be understood as a polypeptide, which is modified to create a protein variant. The parent polypeptide may be a naturally occurring (wild-type) polypeptide or it may be a variant thereof prepared by any suitable means. For instance, the parent polypeptide may be a variant of a naturally occurring polypeptide which has been modified by substitution, chemical modification, deletion or truncation of one or more amino acid residues, or by addition or insertion of one or more amino acid residues into the amino acid sequence of the naturally-occurring polypeptide.

The term “variant” is in the context of the present invention to be understood as a polypeptide which has been modified as compared to a parent polypeptide at one or more amino acid residues.

The term “modification(s)” or “modified” is in the context of the present invention to be understood as to include chemical modification of a polypeptide as well as genetic manipulation of the DNA encoding a polypeptide. The modification(s) may be replacement(s) of the amino acid side chain(s), substitution(s), deletion(s) and/or insertions in or at the amino acid(s) of interest. Thus the term “modified polypeptide” is to be understood as a polypeptide which contains modification(s) compared to a parent polypeptide.

The term “coding sequence” refers in the context of the present invention to the polynucleotide sequence in DNA or RNA that specifies a polypeptide sequence.

The term “upstream” refers in the context of the present invention to polynucleotide sequences located on the proximal site of any given point in relation to the direction of transcription.

The term “downstream” refers in the context of the present invention to polynucleotide sequences located on the distal site of any given point in relation to the direction of transcription.

The term “disulfide bond” refers in the context of the present invention to the covalent bond formed between the sulphur atoms of two Cysteine residues in a polypeptide.

The term “antimicrobial polypeptide or AMP of interest” refers in the context of the present invention to the AMP(s) which is screened for and/or tested by a method of the present invention.

### DETAILED DESCRIPTION OF THE INVENTION

Antimicrobial Polypeptides

The present invention relates to a method for screening a polynucleotide sequence encoding an antimicrobial polypeptide (AMP) and/or testing the antimicrobial activity of an antimicrobial polypeptide according to its ability to kill or inhibit the proliferation of an indicator cell. The present invention also relates to a method for screening a library of polynucleotide sequences encoding one or more antimicrobial polypeptides. Because of the genetic degeneracy the library of polynucleotide sequences may encode only one antimicrobial polypeptide; however it may particularly encode two or more different antimicrobial polypeptides.

In the following reference to the AMP(s) is also to be understood as referring to the polynucleotide sequence(s) encoding said AMP(s). The AMP(s) and the polynucleotide sequence(s) encoding said AMP(s) screened for and/or tested by the present invention may be a variant of a parent AMP, such as a variant generated by manipulation of the nucleotide sequence encoding the parent AMP, and/or it may be a so-called wild-type AMP, i.e. a naturally occurring AMP. The AMP may also be an artificial AMP, i.e. an AMP encoded by a polynucleotide sequence, wherein the polynucleotide sequence is e.g. generated by polynucleotide synthesis.

The AMP(s) and the polynucleotide sequence(s) encoding said AMP(s) screened for and/or tested in the present invention may be obtained from plants, invertebrates, insects, amphibians or mammals, or from microorganisms such as bacteria and fungi. For purposes of the present invention, the term “obtained from” as used herein shall mean that the polypeptide encoded by the nucleotide sequence is produced by a cell in which the nucleotide sequence is naturally present or into which the nucleotide sequence has been inserted. In a particular embodiment, the polypeptide is secreted extracellularly.

In a particular embodiment of the present invention the AMP(s) may be a bacterial polypeptide. For example, the polypeptide(s) may be a gram positive bacterial polypeptide such as a Bacillus polypeptide, e.g., a Bacillus alkalophilus, Bacillus amyloliquefaciens, Bacillus brevis, Bacillus circulans, Bacillus coagulans, Bacillus lautus, Bacillus lentus, Bacillus licheniformis, Bacillus megaterium, Bacillus stearothermophilus, Bacillus subtilis, or Bacillus thuringiensis polypeptide; or a Streptomyces polypeptide, e.g., a Streptomyces lividans or Streptomyces murinus polypeptide; or a gram negative bacterial polypeptide, e.g., an E. coli or a Pseudomonas sp. polypeptide. In another embodiment it may be from a gram negative bacteria.

In another embodiment the AMP(s) and/or the polynucleotide sequence(s) encoding said AMP(S) screened for and/or tested by the present invention may be a fungal polypeptide, and particularly a yeast polypeptide such as a polypeptide obtained from Candida, Kluyveromyces, Pichia, Saccharomyces, e.g. S. carlsbergensis, S. cerevisiae, S. diastaticus, S. douglasii, S. kluyveri, S. norbensis or S. oviformis, Schizosaccharomyces, or Yarrowia polypeptide; or more particularly a filamentous fungal polypeptide such as an Acremonium, Aspergillus, Aureobasidium, Cryptococcus, Filibasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, or Trichoderma polypeptide.

In another particular embodiment, the polypeptide may be obtained from Aspergillus aculeatus, Aspergillus awamori, Aspergillus foetidus, Aspergillus japonicus, Aspergillus nidulans, Aspergillus niger, Aspergillus oryzae, Fusarium bactridioides, Fusarium cerealis, Fusarium crookwellense, Fusarium culmorum, Fusarium graminearum, Fusarium graminum, Fusarium heterosporum, Fusarium longypes, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Trichoderma harzianum, Trichoderma koningii, Trichoderma longibrachiatum, Trichoderma reesei, or Trichoderma viride polypeptide.

Other examples of fungi where the AMP(s) may be obtained from include Pseudoplectania, e.g. P. vogesiaca or P. nigrella, Plectania, e.g. P. melaena, P. melastoma or P. nannfeldti, Umula, e.g. U. helvelloides or Galiella, e.g. G. rufa.

It will be understood that for the aforementioned species, it encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen und Zellkulturen GmbH (DSM), Centraalbureau Voor Schimmelcultures (CBS), and Agricultural Research Service Patent Culture Collection, Northern Regional Research Center (NRRL).

Furthermore, the antimicrobial polypeptide(s) may be identified and obtained from other sources including microorganisms isolated from nature (e.g., soil, composts, water, etc.). Techniques for isolating microorganisms from natural habitats are well known in the art. The polynucleotide sequence(s) may then be derived by similarly screening a genomic or a cDNA library of another microorganism.

AMPs' often exert their antimicrobial effect on the target cell by interacting/binding/sequestering essential molecular targets in said cell. The antimicrobial polypeptide(s) (AMP) and the polynucleotide sequence(s) encoding said AMP(s) screened for and/or tested by the present invention may be a membrane-active antimicrobial polypeptide, or an antimicrobial polypeptide affecting/interacting with intracellular targets, e.g. interacting/binding with molecules involved in signal transduction and/or interacting/binding to cell DNA. The antimicrobial polypeptide(s) may act on cell membranes of the indicator cell, e.g. through non-specific binding to the membrane, usually in a membrane-parallel orientation, interacting only with one face of the bilayer.

The AMP(s) and the polynucleotide sequence(s) encoding said AMP(s) screened and/or tested by a method of the present invention may be an enzyme or a short peptide (less than 100 amino acid residues).

Examples of antimicrobial enzymes include a muramidase, a lysozyme, a protease, a lipase, a phospholipase, a chitinase, a glucanase, a cellulase, a peroxidase, or a laccase. Alternatively, a consortium of enzymes synthesizing conventional antibiotics, e.g. polyketides or penicillins, can be employed.

The AMP(s) and the polynucleotide sequence(s) encoding said AMP(s) screened and/or tested by a method of the present invention may also be a relatively short polypeptide, consisting of less than 100 amino acid residues, typically 5-95 residues, such as between 5-90, 5-80, 5-70, 5-60, 5-50, 5-40, 5-30, 10-90, 10-80, 10-70, 10-60, 10-50, 10-40, 10-30, 20-90, 20-80, 20-70, 20-60, 20-50, 20-40, 30-90, 30-80, 30-70, 30-60, 30-50, 40-90, 40-80, 40-70, 40-60, 50-90, 50-80, 50-70, 60-90 or 60-80 residues. In a particular embodiment of the present invention the AMP(s) and the polynucleotide sequence(s) encoding said AMP(s) to be screened for and/or tested has a low cytotoxicity against normal mammalian cells, including human cells.

Many known short AMPs are cationic and/or hydrophobic polypeptides. Thus it may typically contain several arginine and lysine residues and/or not contain any or very few glutamic acid or aspartic acid residues, and it may often contain a large proportion of hydrophobic residues, such Alanine, Valine, Leucine, Isoleucine, Methionine, Proline, Phenylalanine and Tryptophan.

Furthermore, many known short AMPs generally have an amphiphilic structure, with one surface being positive and the other hydrophobic.

Antimicrobial polypeptides typically has a structure belonging to one of five major classes: alpha-helical, cystine-rich (defensin-like), beta-sheet, an unusual composition of regular amino acids, and containing uncommon modified amino acids.

Examples of AMPs with an alpha-helical structure, which may be used as parent AMPs and/or tested by the method of the present invention include Magainin 1 and 2 which may be obtained from frog skin; Cecropin A, B and P1; CAP18; Andropin which may be obtained from insect hemolymph (e.g. Drosophila melanogaster); Clavanin A which may be obtained from tunicate leukocytes (Styela clava) or Clavanin AK; Styelin D which may be obtained from tunicate leukocytes (Styela clava) and Styelin C; and Buforin II which may be obtained from Asian toad (Bufo bufo garagrizans). Examples of cystine-rich polypeptides include alpha-Defensin HNP-1 (human neutrophil peptide) HNP-2 and HNP-3; beta-Defensin-12, Drosomycin, gamma1-purothionin, and Insect defensin A. Another example is Novispirin G10 which is an alpha-helical antimicrobial polypeptide that does not contain any disulfide bonds. Novispirin G10 (SEQ ID No. 17 in WO 02/00839) is obtained by rational design based on homology to SMAP-29, an ovine cathelicidin peptide.

Examples of beta-sheet polypeptides include Lactoferricin B, Tachy-plesin I, and Protegrin which may be obtained from porcine neutrophils, e.g. Protegrin PG1-5. Examples of polypeptides with an unusual composition include Indolicidin; PR-39 which may be obtained from Porcine leukocytes; Bactenicin Bac5 which may be obtained from sheep leukocytes (Ovis aries) or from bovine lekocytes (Bos taurus) and Bac7 which may be obtained from sheep leukocytes (Ovis aries) or from bovine lekocytes (Bos taurus); and Histatin 5 which may be obtained from human saliva or a variant of Histatin 5, such as d-histatin5 (d-h5) which corresponds to the 14 C-terminal amino acid residues of histatin5, or dhvarl which is a variant of d-h5. Examples of polypeptides with unusual amino acids include Nisin, Gramicidin A, and Alamethicin.

Other examples of AMPs and the polynucleotide sequence(s) encoding said AMP(s) which may be used in the present invention include an antifungal protein (AFP), such as an AFP obtained from Aspergillus, e.g. A. niger or A. giganteus; Defencin hBD3 which may be obtained from human epithelia; Heliomycin which may be obtained from insect hemolymph; Enterocin L50B which may be obtained from Lactobacillus; Thanatin which may be obtained from insect hemolymph; Indolicidin which may be obtained from bovine neutrophils; Tritrpticin which may be obtained from porcine neutrophils; Temporin B which may be obtained from frog skin (e.g. Rana temporaria); Dermaseptin which may be obtained from frog skin; Apidaecin which may be obtained from frog skin or honeybee; SMAP29 which may be obtained from sheep leukocytes (Ovis aries); LL-37 which may be obtained from human or rabbit neutrophils; BPI which may be obtained from human white blood cells; or Plectasin which may be obtained from Pseudoplectania nigrella (e.g. SEQ ID NO:2 in PA 2001 01732).

In a particular embodiment of the invention the AMP screened for and/or tested by a method of the present invention is free of any protecting scaffold proteins.

Library of Polynucleotide Sequences

In one embodiment of the present invention the polynucleotide sequence screened in the present invention may be a library of polynucleotide sequences, such as a library containing two or more different polynucleotide sequences, such as more than 5 different polynucleotide sequences, or more than 10 different polynucleotide sequences, or more than 25 different polynucleotide sequences, or more than 50 different polynucleotide sequences, or more than 100 different polynucleotide sequences, or more than 300 different polynucleotide sequences, or more than 500 different polynucleotide sequences, or more than 1000 different polynucleotide sequences or more than 5000 different polynucleotide sequences or more than 10.000 different polynucleotide sequences, e.g. between 2-10.000 different polynucleotide sequence or between 2-5.000 different polynucleotide sequences or between 2-500 different polynucleotide sequences or between 50-500 different polynucleotide sequences. The library of polynucleotide sequences encoding antimicrobial peptides may be obtained from naturally occurring genomic DNA, cDNA derived from naturally occurring organism or it may be chemically synthesized. Said genomic DNA or cDNA may be derived from any organism, such as one of those described above.

The commercial utility of antimicrobial polypeptides generally depends on their potency, species specificity and ability to perform under the appropriate conditions. Often these conditions are quite different from those under which the polypeptide originally evolved. Most antimicrobial polypeptides have, for example, not been evolved to simultaneously target a broad range of different microbes, to work in a physiological salt range, to evade the human immune system or resist the clearing capacity of the mammalian circulatory system. Therefore it may be desirable to alter one or more properties of a known AMP to improve its performance under a given commercial condition.

Thus in one embodiment of the present invention the library of polynucleotide sequences may be a library of polynucleotide sequences encoding variants of a parent AMP. Said variants may be created by any method known within the art of manipulating polynucleotide sequences, e.g. it may be created by random mutagenesis, by site-directed mutagenesis or by gene shuffling. The sequences to be shuffled may be related sequences from different organisms (so-called “family shuffling”), or they may include a parent sequence and a variant thereof.

In a particular embodiment of the invention random mutagenesis is achieved by shuffling of homologous DNA sequences in vitro such as described by Stemmer (Stemmer, 1994. Proc. Natl. Acad. Sci. USA, 91:10747-10751; Stemmer, 1994. Nature 370:389-391) and/or Crameri (Crameri A., et al., 1997. Nature Bio-technology 15:436-438). The method relates to shuffling homologous DNA sequences by using in vitro PCR techniques. The above method is also described in WO 95/22625 in relation to a method for shuffling homologous DNA sequences. An important step in the method is to cleave the homologous template double-stranded polynucleotide into random fragments of a desired size followed by homologously reassembling the fragments into full-length genes.

In another particular embodiment of the invention random mutagenesis is achieved by the method described in WO 98/41653, which discloses a method of DNA shuffling in which a library of recombined homologous polynucleotides is constructed from a number of different input DNA templates and primers by induced template shifts during in vitro DNA synthesis. In this context especially the special version of this in vitro recombination through induced template shifts during DNA synthesis, described in WO 98/41653, may particularly be used. Here, small (>5 nucleotides) random DNA primers are employed to randomly initiate DNA synthesis on the mutant DNA templates that are to be combined.

Especially if the AMP is a short polypeptide, such as between 20-100 amino acid residues, special attention has to be taken into consideration when using each of the above methods for generation and combination of sequence diversity. Since most shuffling methods rely on a substantial number of identical bp (20-100 bp) flanking the mutations that has to be recombined, the mutations in small polynucleotide sequences are technically difficult to combine by the above described methods.

Accordingly, other formats of directed evolution may be employed on small polynucleotide sequences. In a particular embodiment involving the combination of variants of a given peptide of less than approximately 50 amino acids, one degenerate DNA primer harbouring all the desired mutations may be synthesized. In a given position in this degenerate primer, both the wildtype (wt) (naturally occurring) nucleotide as well as the mutant nucleotide should be present. The frequency of wt-to-mutant nucleotides may be adjusted as considered optimal and rules and considerations to determine the optimal frequency are known in the art. By including all desired mutations in one primer, the desired sequence-space could be completely sampled. This method allows for the sampling and combination of all desired mutations irrespectively of how close they would be in the primary gene sequence.

If polypeptides of more than approximately 50 amino acids are employed, two or more separate and degenerate primers may have to be used. This is due to the constraints generally experienced when synthesizing DNA primers; only DNA primers of less than approximately 180-200 nucleotides may routinely be synthesized.

In another embodiment where polypeptides of more than approximately 50 amino acids are employed, the sequence diversity (the individual mutants) to be combined can individually be harboured in small oligonucleotides of 20-30 base pairs of length. In this approach, a specific DNA oligonucleotide is employed for each mutation that should be included in the library. The mutations may in particular be located in the middle of the small oligonucleotide to optimize annealing. Spiking in several or numerous of these small oligonucleotides in a PCR reaction using the wt polypeptide sequence as backbone for the amplification, would allow for the combination of the desired mutations. By varying the amount of the individual oligonucleotides to be combined, desired ratios of individual variants to wt's can be created. As approximately 10 base pairs are required on each side of the sequence mismatch, this method cannot efficiently combine mutations that are immediately adjacent.

In a particular embodiment doped oligonucleotides may be used as primers for the PCR. Doped oligonucleotides contain mixed bases in un-equal representation to encode the template with different amino acid residues with a specific distribution.

The present invention is not limited to screening libraries of polynucleotide sequences encoding variants of existing or already characterized antimicrobial polypeptides. It may also be used to screen for polynucleotide sequences encoding new and/or unknown antimicrobial polypeptides. For example the library of polynucleotide sequences may comprise polynucleotide sequences obtained from a single microorganism or it may comprise polynucleotide sequences obtained from two or more different microorganisms. Examples of organisms from which the polynucleotide sequence(s) encoding an AMP may be obtained are given above.

Host Cells

The host cell according to the definition may be any cell susceptible to transformation, transfection or infection with a nucleic acid construct. One advantage of the present invention is that the host cell is sensitive to the AMP activity, but that the host cell is first allowed to proliferate in the absence of the AMP (as expression of the AMP is not induced) and then subsequently the host cells are induced to express the AMP. By allowing the host cells to proliferate before induction of AMP expression it is possible to have enough host cells to produce enough AMP to affect an indicator cells. If on the other hand the host cells were not allowed to proliferate before induction of AMP expression it is most likely that the expressed AMP would kill or inhibit the proliferation of the host cells before they were able to express enough amounts of AMP to affect an indicator cell.

The sensitivity of the host cell towards the AMP may be tested by growing the host cells into which a nucleic acid construct comprising the polynucleotide sequence encoding the AMP on for example solid media in the presence and absence, respectively of the inducer and then compare if cell growth of the host cells are inhibited or reduce when the inducer is present as compared when the inducer is absent (as described in example 2 and 3). If cell growth is inhibited or reduced the host cells are sensitive to the peptide when they are expressing it. Another way of testing this is as described in example 13. The host cells expressing the antimicrobial polypeptide are grown in liquid media in the presence and absence, respectively of the inducer. The growth curves of the cultures are monitored for a period of time, such as overnight by measuring the OD and the percentage of growth inhibition is calculated. If the growth of the host cells is reduced or inhibited the host cells are sensitive to the AMP when they are expressing it.

It is an advantage if the host cell does not itself contain and/or express polynucleotide sequence(s) encoding antimicrobial polypeptide(s) as this may interfere with the screening method. This cell characteristic may either be a natural feature of the cell or it may be obtained by deletion of such sequences as described e.g. in Christiansen et al. (1997) or Stoss et al. (1997).

The host cell may be a unicellular microorganism, such as a prokaryote, or a non-unicellular microorganism, such as a eukaryote.

Examples of unicellular cells are bacterial cells such as gram positive bacteria including, but not limited to, a Bacillus cell, e.g., Bacillus alkalophilus, Bacillus amyloliquefaciens, Bacillus brevis, Bacillus circulans, Bacillus clausii, Bacillus coagulans, Bacillus lautus, Bacillus lentus, Bacillus licheniformis, Bacillus megaterium, Bacillus stearothermophilus, Bacillus subtilis, and Bacillus thuringiensis; or a Streptomyces cell, e.g., Streptomyces lividans or Streptomyces murinus, or gram negative bacteria such as E. coli and Pseudomonas sp. In a particular embodiment, the bacterial host cell is Bacillus subtilis

The host cell may also be a eukaryote, such as a mammalian, insect, plant, or fungal cell. In a particular embodiment, the host cell may be a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra).

In a more particular embodiment, the fungal host cell is a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No. 9, 1980).

Examples of yeast host cell include Candida, Hansenula, Kluyveromyces, e.g. K. lactis, Pichia, Saccharomyces, e.g. S. carlsbergensis, S. cerevisiae, S. diastaticus, S. douglasii, S. kluyveri, S. norbensis or S. oviformis, Schizosaccharomyces, or Yarrowia cell, e.g. Y.lipolytica.

In another embodiment the fungal host cell may be a filamentous fungal cell. “Filamentous fungi” include all filamentous forms of the subdivision Eumycota and Oomycota (as defined by Hawksworth et al., 1995, supra). The filamentous fungi are characterized by a mycelial wall composed of chitin, cellulose, glucan, chitosan, mannan, and other complex polysaccharides. Vegetative growth is by hyphal elongation and carbon catabolism is obligating aerobic. In contrast, vegetative growth by yeasts such as Saccharomyces cerevisiae is by budding of a unicellular thallus and carbon catabolism may be fermentative.

Examples of filamentous fungal host cell include a cell of Acremonium, Aspergillus, e.g. A. awamori, A. foetidus, A. japonicus, A. nidulans, A. niger or A. oryzae, Fusarium, e.g. F. bactridioides, F. cerealis, F. crookwellense, F. culmorum, F. graminearum, F. graminum, F. heterosporum, F. negundi, F. oxysporum, F. reticulatum, F. roseum, F. sambucinum, F. sarcochroum, F. sporotrichioides, F. sulphureum, F. torulosum, F. trichothecioides, or F. venenatum, Humicola, e.g. H. insolens, H. lanuginose, Mucor, e.g. M. miehei, Myceliophthora, e.g. M. thermophila, Neurospora, e.g. N. crassa, Penicillium, e.g. P. purpurogenum, Thielavia, e.g. T. terrestris, Tolypocladium, or Trichoderma, e.g. T. harzianum, T. koningii, T. Iongibrachiatum, T. reesei, or T. viride.

In another embodiment of the invention the host cell is a bacterial cell or a eukaryotic cell. Further the bacterial cell may particularly be an ElectroMAX DH10B cell (GibcoBR/Life technologies, UK) or of the genus E. coli, e.g. SJ2 E. coli of Diderichsen et al. (1990) or E.coli with the genotype: F mcrA Δ(mrr-hsdRMS-mcrBC) Φ80lacZΔM15 {lacX74 deoR recAl araD139 Δ(araA-leu)7697 ga/U galk rpsL endA1 nupG, also known as the commercially available TOP10 cells from Invitrogen. Other particular host cells may be strains of Bacillus, such as Bacillus subtilis or Bacillus sp. A particular useful eukaryotic cell is a yeast, e.g. S. cerevisae.

For some polypeptides it may be important for their activity that a disulfide bond is formed between two cysteine residues in the polypeptide. In some host cells, e.g. yeast, formation of disulfide bonds often takes place as a natural part of the expression of polypeptides. However, for other types of host cells it doesn't. Thus in a particular embodiment the redox state of the host cell may be such that it allows disulfide bond formation. This is particularly important if the activity of the antimicrobial polypeptide is affected by the presence of disulfide bond(s) in said polypeptide. The host cell may be such that it naturally allows disulfide bond formation or it may harbour one or more mutations that allow disulfide bond formation. For example the host cell may harbour a mutation in the thioredoxin reductase gene (trxB) and/or in the glutathione reductase gene (gor). In particular the host cell may be a K-12 derivative of E.coli harbouring mutations in both the thioredoxin reductase gene and the glutathione reductase gene, such as the commercially available E.coli origami cells from Novagen.

Expression of a Polynucleotide Sequence in a Host Cell

For expression of a polynucleotide sequence in a host cell, a nucleic acid construct is generally generated comprising the polynucleotide sequence encoding a polypeptide together with polynucleotide sequences capable of facilitating expression of the polynucleotide in the host cell. The polynucleotide sequences facilitating expression of a polynucleotide sequence are often known collectively as an expression vector. For expression of the polynucleotide sequence, i.e. production of the polypeptide, the expression vector comprising the polynucleotide sequence encoding a polypeptide is introduced into a host cell which is then cultured under conditions facilitating expression of the polypeptide. Methods for cloning of polynucleotide sequences and introducing expression vectors into host cells are well-known in the art.

In the present invention a polynucleotide sequence or a library of polynucleotide sequences encoding the antimicrobial polypeptide(s) is expressed in a host cell. Expression of said polynucleotide sequence(s) should be under control of an inducible promoter.

The polynucleotide sequence(s) may in the present invention be expressed intracellularly in the host cell, e.g. in the cytoplasm or other intracellular compartments, such as vacuoles or the endoplasmic reticulum (ER), or it may be expressed in the periplasm or secreted by the host cell.

Expression Vectors

The expression vector may be any vector, e.g. a plasmid or a virus, which may conveniently be subjected to recombinant DNA procedures and which can bring about expression of the antimicrobial polypeptide encoded by the polynucleotide sequence in the host cell. The choice of the vector will typically depend on the compatibility of the vector with the host cell into which the vector is to be introduced. The vectors may be linear or closed circular plasmids.

The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The expression vector used in the present invention may in a particular embodiment comprise one or more selectable markers which permit easy selection of transformed/transfected cells. A selectable marker is typically a gene of which the product provides for biocide or viral resistance, resistance to heavy metals, prototrophy to auxotrophs, and the like.

Examples of bacterial selectable markers are the dal genes from Bacillus subtilis or Bacillus licheniformis, or markers which confer antibiotic resistance such as ampicillin, kanamycin, chloramphenicol or tetracycline resistance.

Suitable markers for yeast host cells are ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hygB (hygromycin phosphotransferase), niaD (nitrate reductase), pyrG (orotidine-5′-phosphate decarboxylase), sC (sulfate adenyltransferase), trpC (anthranilate synthase), as well as equivalents thereof.

Particularly for use in an Aspergillus cell are the amdS and pyrG genes of Aspergillus nidulans or Aspergillus oryzae and the bar gene of Streptomyces hygroscopicus.

The vector used in the present invention may contain an element that permits stable integration of the vector into the host cell's genome or autonomous replication of the vector in the cell independent of the genome.

For integration into the host cell genome, the vector may rely on the nucleotide sequence encoding the polypeptide or any other element of the vector for stable integration of the vector into the genome by homologous or non-homologous recombination. Alternatively, the vector may contain additional nucleotide sequences for directing integration by homologous recombination into the genome of the host cell. The additional nucleotide sequences enable the vector to be integrated into the host cell genome at a precise location in the chromosome. To increase the likelihood of integration at a precise location, the integrational elements should particularly contain a sufficient number of nucleotides, such as 100 to 1,500 base pairs, particularly 400 to 1,500 base pairs, and most particularly 800 to 1,500 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleotide sequences. On the other hand, the vector may be integrated into the genome of the host cell by non-homologous recombination.

For autonomous replication, the vector may further comprise an origin of replication enabling the vector to replicate autonomously in the host cell in question. Examples of bacterial origins of replication are the origins of replication of plasmids pBR322, pUC19, pACYC177, and pACYC184 permitting replication in E. coli, and pUB110, pE194, pTA1060, and pAMβ1 (pAMbeta1) permitting replication in Bacillus. Examples of origins of replication for use in a yeast host cell are the 2 micron origin of replication, ARS1, ARS4, the combination of ARS1 and CEN3, and the combination of ARS4 and CEN6. The origin of replication may be one having a mutation which makes its ability to function temperature-sensitive in the host cell (see, e.g., Ehrlich, 1978, Proceedings of the National Academy of Sciences USA 75: 1433). More than one copy of a polynucleotide sequence of the present invention may be inserted into the host cell to increase production of the gene product. An increase in the copy number of the nucleotide sequence can be obtained by integrating at least one additional copy of the sequence into the host cell genome or by including an amplifiable selectable marker gene with the nucleotide sequence where cells containing amplified copies of the selectable marker gene, and thereby additional copies of the nucleotide sequence, can be selected for by cultivating the cells in the presence of the appropriate selectable agent.

The procedures used to ligate the elements described above to construct the expression vectors are well known in the art (see, e.g., Sambrook et al., 1989, supra).

Inducible Promoters and Inducers

The expression vector used to express the polynucleotide sequence encoding an antimicrobial polypeptide according to the present invention should comprise an inducible promoter so that expression of said AMP may be controlled by a regulator. It is an advantage if the promoter allows tight regulation of the synthesis of the encoded AMP, i.e. that leakiness from the promoter is kept at a minimum. In addition, control of the transcription of the encoded AMP may particularly be significant as particularly short polypeptides are often inherently unstable and easily degraded in the cytoplasm of microorganisms. The inducible promoter may be regulated by more than one regulator. For example the promoter may be positively and negatively regulated, respectively, by two different compounds, e.g. in the presence of an inducer, expression from the promoter may be turned on; while in the absence of said inducer only very low levels of expression occur from the promoter. The uninduced (i.e. in the absence of the first inducer) levels may then be further repressed by the presence of a repressor. By varying the activity of the two regulators, protein expression levels may be manipulated to optimize expression of potentially toxic or essential genes.

One example of an inducible promoter and inducers is the Lac promoter as described in Taguchi S., Nakagawa K., Maeno M. and Momose H.; “In Vivo Monitoring System for Structure-Function Relationship Analysis of the antibacterial peptide Apidaecin”; Applied and Environmental Microbiology, 1994, pp. 3566-3572, which may be regulated by presence of the inducer lactose or by the synthetic non-digestible lactose derivative IPTG. Other examples include the trp promoters induced by tryptophan or gal promoters induced by galactose for E. coli, gall promoter for S. cerevisiae, AOX1 promoter for Pichia pastoris, pMT (metallothionein) promoter for Drosophila, MMTV LTR, pVgRXR or pIND promoters for mammalian expression. It is an advantage to use an inducer which is not metabolized or digested in the host cell as this may keep the inducer concentration constant during the screening procedure. Furthermore, it may be an advantage to select/use a promoter for which the corresponding inducer is able to permeate the cell membrane(s) and gain access to the promoter. In a particular embodiment of the invention the promoter may be the pBAD promoter as used in the examples, vide infra, which is induced by the digestible inducer arabinose. If the pBAD promoter is used the host cells ability to digest arabinose may be eliminated by deleting suitable genes from the host cell genome so as to achieve the above mentioned advantage of having a constant level of inducer.

The pBAD promoter is an example of a promoter which is regulated by two regulators as this is both positively and negatively regulated by AraC and cAMP-CRP. In the presence of arabinose, expression from the promoter is turned on, while in the absence of arabinose, only very low levels of expression occur from the promoter.

Uninduced levels may be even further repressed by culturing the cells in the presence of glucose. Glucose acts by lowering cAMP levels, which in turn decreases the binding of cAMP-CRP to the promoter region of pBAD. As cAMP levels are lowered, transcriptional activation is decreased. This is an advantage if the antimicrobial polypeptide of interest is extremely growth inhibitive or toxic to the host. In conclusion, by varying the activity of the two regulators, protein expression levels can be manipulated to optimize expression of potentially toxic or essential genes.

Other Regulatory Sequences

The nucleic acid construct used for expressing the polynucleotide sequence encoding the antimicrobial polypeptide of the present invention may comprise, besides the promoter and the polynucleotide sequence encoding said AMP, other polynucleotide sequences of importance for the expression of said AMP(s). For example the nucleic acid construct may further comprise a signal peptide coding region, a transcription terminator sequence, a leader sequence, a polyadenylation sequence and/or a propeptide coding region.

A signal peptide coding region is a polynucleotide sequence located upstream of the 5′ end of the coding sequence, which is both transcribed and translated and where the translated signal peptide directs the encoded polypeptide into the host cell's secretory pathway. The 5′ end of the coding sequence may inherently contain a signal peptide coding region naturally linked in translation reading frame with the segment of the coding region that encodes the secreted polypeptide. Alternatively, the 5′ end of the coding sequence may contain a signal peptide coding region which is foreign to the coding sequence. The foreign signal peptide coding region may be required where the coding sequence does not naturally contain a signal peptide coding region. Alternatively, the foreign signal peptide coding region may simply replace the natural signal peptide coding region in order to enhance secretion of the polypeptide. However, any signal peptide coding region which directs the expressed polypeptide into the secretory pathway of a host cell of choice may be used in the present invention.

Examples of signal peptide coding regions for bacterial host cells are the signal peptide coding regions obtained from the genes for Bacillus NCIB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are described by Simonen and Palva, 1993, Microbiological Reviews 57: 109-137.

Another example is the g11 signal peptide, as used in the examples, which directs expression to the periplasm.

Examples of signal peptide coding regions for filamentous fungal host cells are the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase.

Useful signal peptide coding regions for yeast host cells are obtained from the genes for Saccharomyces cerevisiae alpha-factor and Saccharomyces cerevisiae invertase. Other useful signal peptide coding regions are described by Romanos et al., 1992, supra.

A transcription terminator sequence is a polynucleotide sequence recognized by a host cell to terminate transcription. The terminator sequence is generally located downstream of the 3′ terminus of the coding sequence.

Examples of terminators for filamentous fungal host cells include terminators obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease.

Examples of terminators for yeast host cells include terminators obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

A leader sequence is in the context of the present invention to be understood as a polynucleotide sequence which is transcribed but not translated and which contains the ribosome-binding site. The leader sequence is linked to the 5′ terminus of the coding sequence. Examples of leader sequences for filamentous fungal host cells include those obtained from the genes for Aspergillus oryzae TAKA amylase and Aspergillus nidulans triose phosphate isomerase.

Examples of leader sequences for yeast host cells include those obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

A polyadenylation sequence is a polynucleotide sequence linked to the 3′ end of the coding sequence and which, when transcribed, is recognized by the host cell as a signal to add polyadenosine residues to the transcribed mRNA.

Examples of polyadenylation sequences for filamentous fungal host cells include those obtained from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase.

Useful polyadenylation sequences for yeast host cells are described by Guo and Sherman, 1995, Molecular Cellular Biology 15: 5983-5990.

A propeptide region is a part of the coding sequence which encodes an amino acid sequence positioned at the amino terminus of a polypeptide. A polypeptide comprising a propeptide region is known as a propolypeptide or a proenzyme (or a zymogen in some cases). A propolypeptide is generally inactive and can be converted to a mature active polypeptide by catalytic or autocatalytic cleavage of the propeptide from the propolypeptide. The propeptide coding region may be obtained from the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila laccase (WO 95/33836). If both a signal peptide coding region and a propeptide region are present at the amino terminus of a polypeptide, the propeptide region may be positioned next to the amino terminus of the mature polypeptide and the signal peptide region may be positioned next to the amino terminus of the propeptide region. In this context the term “mature polypeptide” refers to the functional active polypeptide without the propeptide sequence.

Introduction of Nucleic Acid Constructs in a Host Cell

The polynucleotide sequence encoding the antimicrobial polypeptide in the present invention may be introduced into the host cell by any method and methods for introducing nucleic acid constructs into host cells are well known to a person skilled in the art.

For example a nucleic acid construct may be introduced into a bacterial host cell may by protoplast transformation (see, e.g., Chang and Cohen, 1979, Molecular General Genetics 168: 111-115), using competent cells (see, e.g., Young and Spizizin, 1961, Journal of Bacteriology 81: 823-829, or Dubnau and Davidoff-Abelson, 1971, Journal of Molecular Biology 56: 209-221), electroporation (see, e.g., Shigekawa and Dower, 1988, Biotechniques 6: 742-751), or conjugation (see, e.g., Koehler and Thorne, 1987, Journal of Bacteriology 169: 5771-5278). Fungal cells may be transformed by a process involving protoplast formation, transformation of the protoplasts, and regeneration of the cell wall in a manner known per se. Suitable procedures for transformation of Aspergillus host cells are described in EP 238 023 and Yelton et al., 1984, Proceedings of the National Academy of Sciences USA 81: 1470-1474. Suitable methods for transforming Fusarium species are described by Malardier et al., 1989, Gene 78: 147-156 and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920.

Indicator Cells

The indicator cell used in the present invention may be any cell which it is interesting to test for antimicrobial polypeptide activity against. Thus the indicator cell may be any cell which is sensitive to an antimicrobial polypeptide activity. One advantage of the present invention is that the antimicrobial activity is tested on indicator cells thus one need only to introduce the polynuleotide sequence encoding said AMP activity into a single type of host cells but is able to test the AMP activity on a number of different indicator cells. Thus it is possible to test the antimicrobial activity towards several indicator cells, i.e. a library of indicator cells without having to introduce the polynucleotide sequence encoding said AMP(s) into more than one host cell. It is thereby possible to reduce the amount of work which is associated with introducing a polynucleotide sequence into host cells.

The indicator cell may be a prokaryotic cell, e.g. a bacterium, or it may be a eukaryotic cell, such as a fungal cell, a plant cell, an insect cell or a mammalian cell. Examples of fungal cells include both filamentous fungi and yeast.

For example an indicator cell may be any of those described above.

Examples of useful bacterial indicator cells include Bacillus, e.g. B. subtilis or the chloramphenicol-resistant B. subtilis (1315-1), Bordetella, e.g. B. bronchiseptica, Burkholderia, e.g. B. cepacia, Coagulase-negative Staphylococcus, Staphylococcus carnosus, Citrobacter, e.g. C. freundii, Enterococcus, e.g. E. hirae or E. spec., Escherichia, e.g. E. coli such as the TOP10 E.coli cells from Invitrogen, Klebsiella, e.g. K. pneumonia, Micrococcus, e.g. M. luteus, Mycobacterium, e.g. M. smegmatis, Pseudomonas, e.g. P. aeruginosa, Staphylococcus, e.g. S. aureus, S. epidermidis or S. simulans or Stenotrophomonas, e.g. S. maltophila.

Examples of yeasts which may be used as indicator cells include Saccharomyces, e.g. S. cerevisiae, Candida, e.g. C. albicans or Pityrosporum.

Examples of filamentous fungi which may be used as indicator cells include Epdidermophyton, e.g. E. floccosum, Trichophyton, e.g. T. mentagrophytes or Aspergillus, e.g. A. niger, Fusarium, e.g. Flongypes.

In a particular embodiment the methods of the present invention may be used to test a library of indicator cells, i.e whether two or more indicator cells are sensitive towards a particular antimicrobial polypeptide or towards a library of polynucleotide sequences encoding one or more, e.g. a library of antimicrobial polypeptides. In particular more than 5 indicator cells, such as more than 10 indicator cells, or more than 25 indicator cells, or more than 50 indicator cells, or more than 100 indicator cells or more than 200 indicator cells, or more than 300 indicator cells or more than 500 indicator cells or more than 1000 indicator cells or more than 5000 indicator cells may be used in a method of the present invention. For example 2-5000 indicator cells, e.g. 2-1000 indicator cells or 2-500 indicator cells or 2-100 indicator cells or 5- 1000 indicator cells or 5-500 indicator cells or 5-100 indicator cells or 10-1000 indicator cells or 10-500 indicator cells or 10-100 indicator cells or 50-500 indicator cells may be tested in a method of the present invention. Typically if a library of indicator cells are used in the present invention the indicator cells are cultured separately in the presence of the antimicrobial polypeptide(s) or the host cells expressing the antimicrobial polypeptide(s).

Screening Process

As mentioned above the present invention relates to a method for screening a polynucleotide sequence encoding an antimicrobial polypeptide, said method comprising the steps of:
a) introducing the polynucleotide sequence in a host cell, wherein expression of said polynucleotide is under control of an inducible promoter and wherein said host cell is sensitive to the antimicrobial peptide encoded by the polynucleotide sequence
b) cultivating the host cells of in the absence of an inducer capable of inducing expression of the antimicrobial peptide
c) cultivating the host cells in the presence of an inducer capable of inducing expression of the antimicrobial peptide
d) cultivating the host cells in the presence of an indicator cell, wherein said indicator cell is sensitive to the antimicrobial polypeptide encoded by the polynucleotide sequence
e) selecting host cells capable of reducing the proliferation of indicator cells
f) recovering the polynucleotide sequence encoding an antimicrobial polypeptide from the host cells selected in step e).

Prior to step a) of the screening process certain preparatory steps may be necessary. An indicator cell for which an antimicrobial polypeptide with a killing and/or cell proliferation inhibiting effect it is desired to identify should be found, examples of useful indicator cells are given above. A suitable host cell and an expression vector compatible with said host cell should be chosen, examples of suitable host cells and vectors are given above. In a particular embodiment it may be a host cell, which allows formation of disulfide bond, e.g. it may be a host cell harbouring a mutation which alters the redox state of the host cell, such as a host cell which harbours a mutation in the thioredoxin reductase gene and/or a mutation in the glutathione reductase gene. In one embodiment of the method the polynucleotide sequence is a library of polynucleotide sequences, and in this case a library of polynucleotide sequences should be prepared prior to step a). Thus a library of polynucleotide sequences is screened by the method. Examples of libraries of polynucleotide sequences are given above, such as a sample of polynucleotide generated by mutating a parent polynucleotide sequence encoding a parent antimicrobial polypeptide, or a sample of polynucleotide sequences representing the genome or polypeptides expressed by a specific organism or cell, or by two or more organisms or cells. Said library may be prepared by any method, e.g. any conventional method known to a person skilled in the art.

As part of introducing the polynucleotide sequence into a host cells, i.e. in step a), said host cells may be separated on the basis of e.g. if a polynucleotide sequence has actually been introduced into the host cell and/or if the host cell is viable and/or its proliferation has not been inhibited. This may typically be done by the use of a selection marker. Examples of suitable selection markers are given above. However, it is not essential to the screening method to separate non-viable host cells from viable host cells as the antimicrobial polypeptide is selected on the basis of its killing or cell proliferation inhibiting effect on the indicator cell. If a selection marker is used the host cells may typically be sub-cultured in the absence of the selection marker, as the selection marker may kill and/or inhibit proliferation of the indicator cell. Instead of sub-culturing the host cell a compound capable of inactivating the selection marker may be added to the culture. Methods for culturing and/or sub-culturing host cells are known to a person skilled in the art and include culturing on solid media, e.g. a conventional agar plate or culture in a liquid media, e.g. in a shake or a microtiter plate.

In step b) the host cells are cultivated in the absence of the inducer so that said cells can proliferate without being killed or its proliferation being inhibited by the antimicrobial polypeptide to which they are sensitive. The host cells may by cultured in the presence of any suitable nutrient medium using methods known in the art. For example, the cell may be cultivated by shake flask cultivation, on agar plates, in microtiter plates or small-scale fermentation. Typically a suitable nutrient medium comprises carbon and nitrogen sources and inorganic salts and may be available from commercial suppliers or be prepared according to published compositions (e.g., in catalogues of the American Type Culture Collection). The host cells may be cultivated in a suitable medium without an inducer for as long as necessary, typically this may be for 10-24 hours, 10-36 hours, 1048 hours, 10-72 hours, such as 10 hours, 24 hours, 48 hours or 72 hours.

In step c) the host cells are cultivated in the presence of an inducer so as to induce expression of the antimicrobial polypeptide. Typically said cells are cultivated in the same nutrient medium as in step b) but with the exception that it now also contains the inducer. A suitable nutrient medium, method of cultivation and time of cultivation may be as described above. The amount of inducer depends on the type of inducer and type of inducible promoter and is well known to a person skilled in the art. For example if arabinose is used as inducer it may typically be used in the concentration range from 0.0001-20% arabinose, e.g. 0.001-10% or 0.01-1% arabinose and if IPTG is used as inducer it may typically be used in range of 0.001-1 mM IPTG. The presence of the inducer should induce expression of the antimicrobial polypeptide by the host cells and thereby kill or inhibit the proliferation of said host cells. The dose of inducer may be regulated to control for example whether the host cells are completely killed or only their proliferation is inhibited and/or it may be varied during the time period of cultivation. If the dose of inducer is varied during cultivation it may e.g. be lower in the beginning to induce expression of the antimicrobial polypeptide but only slightly affect the proliferation of the host cells and then increased to eventually kill the host cells.

In one embodiment of the invention the host cells may be lysed before step d), i.e. after they have had time to produce the AMP but before culturing them in the presence of an indicator cell and/or before culturing the indicator cell(s) in the presence of the AMP. The AMP may by itself lyse the cells or this may be performed by affecting the host cells by external factors capable of lysing of cells, such as addition of chloroform or lysozym, infection with a lytic phage or the host cells may be lysed by exposing them to sonication, high temperature or an acidic pH or by a combination of two or more factors. For example the host cells may be lysed by exposing them to at least 60 degrees C., such as at least 70 degrees C., or at least 80 degrees C., or at least 90 degrees C. or at least 95 degrees C., e.g. between 65 and 95 degrees C., or between 75 and 95 degrees C., or between 75 and 85 degrees C. The host cells may also be lysed by exposing them to an acidic pH, such as below pH 6, or below pH 5 or below pH 4 or below pH 3, or below pH 2, e.g. between pH 2 and pH 5, or between pH 2 and pH 4, or pH between pH 2.5 and 3. In a particular embodiment of the present invention the host cells are lysed by a exposing them to a high temperature and an acidic pH, e.g. a temperature between 65 and 95 degrees C. and a pH between pH 2 and pH 6, particularly a temperature between 75 and 85 degrees C. and a pH between pH 2 and pH 4, more particularly a temperature of 80 degrees C. and a pH between pH 2.5 and 3.

In step d) the host cells are cultivated in the presence of an indicator cell, so that the killing or proliferation inhibiting effect of the antimicrobial polypeptide expressed by the host cell can be tested on the indicator cell. The host cells and indicator cells may be cultivated in any suitable nutrient media, by any suitable method and for any period of time, e.g. as described above. The host cells and the indicator cells may be in direct contact or in indirect contact with each other, e.g. they may be cultivated without direct contact but by a method allowing diffusion of the antimicrobial polypeptide expressed by the host cells to the indicator cells. The viability and/or proliferation of the indicator cells may be tested by any known method, e.g. as described below.

In one embodiment of the invention the effect of the antimicrobial polypeptide expressed by the host cell may be tested on two or more different indicator cells, e.g. the effect of the AMP may be tested on a library of indicator cells. Typically this may be performed by cultivating the each of the different indicator cells separately in the presence of the host cells expressing the AMP(s) to distinguish the indicator cells from each other.

Host cells which express an antimicrobial polypeptide capable of killing or reducing the proliferation of the indicator cells are identified in step e). Different criteria for selecting the host cell(s) may be used. For example only host cells which greatly affect the indicator cells may be selected, such as host cells expressing an antimicrobial polypeptide capable of completely killing the indicator cell, or all host cells capable of affecting the proliferation rate of the indicator cell may be selected, e.g. all host cells are selected independent of whether they kill the indicator cell, inhibit the proliferation of the indicator cell slightly or inhibit the proliferation of the indicator cell greatly. If the effect of the antimicrobial polypeptide is tested on two or more different indicator cells one may e.g. select only host cells expressing an antimicrobial polypeptide which kills and/or inhibits the proliferation of two or more different indicator cells or one may select all host cells expressing an antimicrobial polypeptide capable of killing or inhibiting the proliferation of at least one type of indicator cell.

In step f) the polynucleotide sequence encoding an antimicrobial polypeptide expressed by a host cell selected in step e) is recovered from said host cell. Recovering of said polynucleotide may be performed by any known method. The polynucleotide sequences may be amplified by conventional methods, e.g. PCR amplification. The identified and amplified polynucleotide sequence may then be inserted into a host cell capable of producing said antimicrobial polypeptide. For example the antimicrobial polypeptide may be expressed through fusion to another polypeptide which then may be exported or secreted by the host cell. In particular the antimicrobial polypeptide may be expressed through fusion to a polypeptide which is larger, i.e. for which the mass is larger, than the antimicrobial polypeptide. Said polypeptide may have the function of protecting the antimicrobial polypeptide of interest from digestion within the cell and thereby inactivation by the host cell enzymes and/or the polypeptide may have the function of lowering the effect of the antimicrobial polypeptide on the host cell so that the host may proliferate and continue expression of the antimicrobial polypeptide without being significantly affected by the expressed antimicrobial polypeptide, an effect which may occur if the antimicrobial polypeptide has not been incorporated into the polypeptide. The identified and amplified polynucleotide sequence encoding the antimicrobial polypeptide may also be mutated as described, vide supra, e.g., by random mutagenesis, by gene shuffling, or by synthesizing degenerate genes. These mutated nucleotide sequences may then be screened again according to steps a) to f) to identify nucleotide sequences encoding new antimicrobial polypeptides with an improved effect e.g. by lowering the concentration of inducer in subsequent screenings. In this context the term “improved effect” refers to that the AMP(s) identified by the second (or more) time the method is repeated has an effect, such as specificity towards indicator cells, activity or stability under given conditions which is better as compared to the same effect of the AMP(s) identified by the previous time the method was performed. A better specificity towards indicator cells(s) may for example be that the number of indicator cells which are sensitive to a given AMP may e.g. be higher or lower. A better activity may for example be that the concentration or amount of a given AMP which is necessary to inhibit proliferation of a given indicator cell may be lower. A better stability may be that a particular AMP is more stabile under e.g. a high temperature, an acidic or alkaline pH or in the presence of certain compounds such as detergents.

In one embodiment of the present invention the screening and/or testing method of the present invention is carried out by application of conventional plate assays so that after introduction of the polynucleotide sequence in the host cell in step a), said cells are streaked out on a plate comprising a nutrient medium without an inducer, but optionally with an antibiotic or another selectable marker. An antibiotic or selectable marker may be used if the expression vector used for construction of the polynucleotide sequence encoding the antimicrobial polypeptide comprises a gene encoding resistance to said antibiotic or selectable marker. The presence of the selectable marker/antibiotic will make only those host cells survive into which an expression vector comprising a polynucleotide sequence has actually entered, i.e. host cells where the transformation, transfection or infection has been successful. In a particular embodiment a filter, such as a cellulose acetate filter is placed on top of the plate with the nutrient medium with or without a selection marker and the host cells are streaked out on the filter. The plate is then incubated for a period of time to enable colony formation of transformed/transfected/infected host cells.

In a particular embodiment of the present invention the host cells are sub-cultured, i.e. from the plate one or more samples of host cells or if the host cells were cultured on a filter said filter comprising the host cells may be transferred to another plate. Said plate comprises a nutrient medium and it may comprise an inducer capable of inducing expression and production of the antimicrobial polypeptide comprised in the inserted expression vector. In a particular embodiment the colonies of host cells may be sub-cultured by transferring them to another plate by placing a filter on top of the plate and then stripping the colonies of host cells of the plate with the filter and transferring the filter to a new plate.

In a particular embodiment the host cells may, instead of being transferred from a plate without inducer to a plate with inducer (i.e. sub-cultured), be overlaid with another layer of e.g. agarose comprising the inducer, where the inducer may be arabinose. The plate is then again incubated for a period of time to allow expression of the antimicrobial polypeptides.

The indicator cells may then be placed on top of the plate comprising the host cells in a suitable nutrient medium and incubated for a period of time allowing the antimicrobial polypeptide to exert its effect on the indicator cell. If the host cells have been cultured on a filter the filter may be removed before placing the indicator cells on top of the plate comprising the host cells. If the host cells have been culture in the presence of a selection marker but they have not been sub-cultured, i.e. the selection marker is still present; a compound capable of degrading/removing the selection marker may be added to the plate before and/or together with the indicator cell.

For example if ampicillin has been used as selection marker beta-lactamase, which is capable of degrading ampicillin, may be added before and/or together with the indicator cells. If a transformed/transfected/infected host cell colony inhibits proliferation of the indicator cell in the area surrounding the host cell colony it may be deduced that said host cell express an antimicrobial polypeptide which inhibits the proliferation or kills the indicator cell. Inhibition of the proliferation of the indicator cell may be detected as described below.

In another embodiment of the present invention the screening and/or testing method may be carried out in a liquid assay. Thus for example after introduction of the polynucleotide sequence into the host cells, said host cells may be seeded in a microtiter plate or a shake flask, at a concentration facilitating growth of individual colonies in each well/flask, in a nutrient medium without an inducer and cultured for a period of time necessary for proliferation of the host cells. Thereafter an inducer may be added to the nutrient medium and the host cells may be cultured for a period of time allowing expression of the AMP. In one embodiment the indicator strain may then be added to the microtiter plate/shake flask and cultures are then again cultured for a period of time allowing the AMP to act on the indicator cells. Finally, proliferation of the indicator cells during this period of time may be measured by, e.g. optical density (OD). In another embodiment the culture media is used to test the antimicrobial activity on an indicator cell in another assay, such as a radial diffusion assay. The host cells may be lysed before using the culture media.

A combination of a solid plate assay and a liquid assay may also be used. For example after introducing the polynucleotide sequence encoding an antimicrobial activity into a host cell, said host cells may be cultured on a solid media, e.g. an agar plate to select for viable cells comprising the polynucleotide sequence and then the subsequent steps may be performed in a liquid media as e.g. described above.

In another embodiment of the present invention the screening and/or testing method may be performed by cloning the polynucleotide sequence encoding an AMP into a phage, for example lambda-ZAP (Stratagene, where expression is driven by the Lac promoter, induced by IPTG) and then infect the host cells (for example E.coli XL-1-Blue) with the packaged phages by e.g. mixing the phages and the host cells on a top agar solution and plate them on a LB plate. The plates are then incubated to prevent lysogenesis for a period of time, e.g. at 42° C. and for 3-4 hours. When tiny plaques are visible the infected host cells are overlaid with an indicator cell and an inducer, such as a strain of Bacillus and IPTG, to induce the expression of the polypeptide. The plates are then incubated for a period of time at e.g. 37° C. The host cells will lyse but they will still have time to produce the AMP before dying. The synthesized polypeptide will be liberated and it may then inhibit proliferation of the indicator cells. After e.g. an overnight incubation inhibition of the proliferation of the indicator cells may be visible as clearing zones surrounding the host cells. For this embodiment it is important that the indicator strain can not be infected by the phage.

The present invention also relates to a method for screening a library of polynucleotide sequences encoding one or more antimicrobial polypeptide(s), comprising:
a) cultivating an indicator cell in the presence of the antimicrobial polypeptide(s), wherein the antimicrobial polypeptide(s) has/have been expressed by a host which is sensitive to said antimicrobial polypeptide(s), and wherein expression of each of the polynucleotide sequences in the library has been under control of an inducible promoter, and wherein the host cells comprising the library polynucleotide sequence has been cultivated in the absence of an inducer and subsequently in the presence of an inducer
b) selecting a host cell expressing an antimicrobial polypeptide which is capable of reducing the proliferation of the indicator cell

The present invention also relates to a method for testing the antimicrobial activity of an antimicrobial polypeptide comprising:
a) cultivating an indicator cell in the presence of the antimicrobial polypeptide(s), wherein the antimicrobial polypeptide(s) has/have been expressed by a host which is sensitive to said antimicrobial polypeptide(s), and wherein expression of each of the polynucleotide sequences in the library has been under control of an inducible promoter, and wherein the host cells comprising the library polynucleotide sequence has been cultivated in the absence of an inducer and subsequently in the presence of an inducer

Both of said methods may further comprise the step of:
c) recovering the polynucleotide sequence encoding the antimicrobial polypeptide from the host cell.

The embodiments described above concerning preparatory steps which may be performed before step a), preparation of a library of polynucleotide sequences encoding one or more antimicrobial polypeptides, preparation of a host cell comprising a polynucleotide sequence encoding an antimicrobial polypeptide, cultivating a host cell comprising said polynucleotide sequence in the absence of an inducer, cultivating a host cell comprising said polynucleotide sequence in the presence of an inducer, and additional steps such as lysing the host cell may also be used in the two above mentioned methods.

The indicator cell may cultivated in the presence of the antimicrobial polypeptide as described above for the previous method by cultivating it directly or indirectly in the presence of the host cell expressing the antimicrobial polypeptide(s). If the indicator cell is cultured in a liquid media the antimicrobial polypeptide(s) may be added to the culture of the indicator cell, e.g. the culture broth into which the host cells have expressed the AMP may be added to the indicator cell. The AMP may be purified before adding it to the indicator cell.

In another embodiment the host cell expressing the AMP(s) has been cultured on solid media, e.g. on a filter placed on agar, and the antimicrobial polypeptide(s) has/have diffused into said solid media; the host cells may subsequently be removed and the indicator cell may be added to the solid media and thereby be cultivated in the presence of the antimicrobial polypeptide. In a particular embodiment of the above two methods a library of indicator cells is cultivated in the presence of the AMP(s). This may in particular be performed by cultivating each of the different indicator cells separately in the presence of the AMP(s). For example if the indicator cells are cultivated in a liquid media, such as in a microtiter plate or a shake flask, each of the different indicator cells may be cultivated as separate culture, e.g. in separate wells of a microtiter plate or separate shake flasks. If the indicator cells are cultured on solid media, they may be cultured as separate colonies or on separate agar plates.

The recovered polynucleotide sequence of step c) may also be further manipulated as described above and the method(s) may be repeated one or more times as described above to identify an AMP with an improved effect.

Methods for Testing Viability and Proliferation Rates

Methods for testing the viability and/or proliferation rate of cells are well known to a person skilled in the art. For example if the cells are cultured on a plate, e.g. an agar plate which may be in the presence of a selection marker such as an antibiotic, the presence of a colony and/or the size of a colony can be used to evaluate the viability and/or the proliferation rate of the cells. Typically cells are spread over an agar plate comprising a selection marker and then the plates of are cultured for a period of time, such as overnight. The presence of visible colonies indicates cells which cells are viable.

The viability and/or proliferation rate of cells cultured in suspension may be measured by measuring the optical density (OD) of the cultures.

Other methods include staining with MTT (3-(3-(4,5-Dimethylthiazol-2-yl)-2,5-diphenyltetrazolium bromide) which stains viable cells blue, Alamar Blue or 2,3,5-triphenyltetrazolium chloride. For example cells may plated on e.g. an agar plate, a microtiter plate or they may grow in suspension and then the dye is added so that it is possible to e.g. visually identify cells which are alive. The amount of dye after a certain culture time may also be used to evaluate the proliferation rate of the cells. For example if the same number of cells have been seeded in e.g. suspension or on a microtiter plate and then allowed to proliferate for a certain period of time, such as 24 hours, the cells may then be stained subsequently and the amount of dye present used to evaluate the proliferation rate of the cells.

A further method for testing viability and proliferation rates is to measure expression of a reporter gene present in the indicator strain, for example the Green Fluorescent Protein (GFP), Luciferase (LUC), beta-Glucoronidase (GUS) or beta-galactosidase (GAL) by the use for example of a Victor Wallac 1420 multilabel counter (Wallac Danmark A/S). The growth of the indicator cells may be correlated with the measurement of the reporter protein present in the culture.

The antimicrobial activity may also be tested in a radial diffusion assays as e.g. described in WO 03/044049A1 based on the protocol published in (Lehrer et al., (1991) Ultrasensitive assays for endogenous antimicrobial polypeptides J Immunol Methods 137: 167-173) with some modifications. Briefly, indicator cells are added to an underlay of agarose into which several holes are made, e.g. by solidifying the agarose on a Nunc omnitray plate containing a nunc-ImmunoTSP PS rack inside to obtain 96 holes in the agarose media. The samples are subsequently added to the holes and incubated for a period of time, e.g. at 37 degrees C for 3 hours to allow for the antimicrobial activity to exert its action on the indicator cell. Next an overlay (LB media with agar) is poured on top of the plate and the plate is incubated again to allow for the antimicrobial activity to exert its action on the indicator cell, e.g. it may be incubated for 10-24 or 10-48 hours to allow the growth of the indicator cells. Antimicrobial activity is detected as bacterial clearing zones around the wells. Any method of staining may be used to distinguish living cells from dead cells as described above.

Use of Antimicrobial Polypeptides

Antimicrobial polypeptides identified by the method of the present invention may be used in a variety of applications, for example it may be used within areas such medical care, cosmetic care, animal feed, cleaning systems or for various industrial applications.

Medical Applications

The invention also relates to the use of an antimicrobial polypeptide found by the present invention as a medicament. Further, an antimicrobial polypeptide or composition comprising said antimicrobial polypeptide found by the present invention may also be used for the manufacture of a medicament for controlling or combating microorganisms, such as fungal organisms or bacteria.

The antimicrobial polypeptide found by the present invention may also be used as an antimicrobial veterinarian or human therapeutic or prophylactic agent. Thus, the antimicrobial polypeptide found by the present invention may be used in the preparation of veterinarian or human therapeutic agents or prophylactic agents for the treatment of a microbial, such as fungal infection, bacterial infection or viral infection; it may also be used for multi-resistant infections.

Examples of infections or diseases were an AMP of the present invention may be used include cystic fibrosis (CF), ventilator-associated pneumonia (VAP), candidiasis, HIV and nasal carriage of S. aureus.

The antimicrobial polypeptide found by the present invention may also be used in wound healing composition or products such as bandages, medical devices such as, e.g., catheters. Thus, the antimicrobial polypeptides found by the present invention may be useful as a disinfectant, for example it may be used for topical application, e.g. for the treatment of acne, infections in the skin, wounds, chronic wounds, bruises and the like. Other examples of infections for which an AMP of the present invention may be used include treatment of infections in the eye or the mouth, and diabetic foot ulcers.

Cosmetic Applications

An AMP of the present invention may for example be used in a variety of different products for personal care such as lotions, creams, gels, ointments, soaps, shampoos, conditioners or oral care products such as mouth wash, in antiperspirants or deodorants; in foot bath salts; for cleaning and disinfection of contact lenses, teeth (oral care), or for anti-dandruff hair products, such as shampoos.

Feed Applications

Another application for an antimicrobial polypeptide identified by the present invention is the employment of said antimicrobial polypeptide in animal feed products. The term animal includes all animals, including human beings. Examples of animals are non-ruminants, and ruminants, such as cows, sheep and horses. In a particular embodiment, the animal is a non-ruminant animal. Non-ruminant animals include mono-gastric animals, e.g. pigs or swine (including, but not limited to, piglets, growing pigs, and sows); poultry such as turkeys and chicken (including but not limited to broiler chicks, layers); young calves; and fish (including but not limited to salmon).

The term feed or feed product means any compound, preparation, mixture, or composition suitable for, or intended for intake by an animal.

It may also be used as an antimicrobial in food products and would be especially useful as a surface antimicrobial in cheeses, fruits and vegetables and food on salad bars. Other uses include preservation of foods, beverages or food ingredients.

Cleaning Systems

Further, it is contemplated that the antimicrobial polypeptides found by the present invention can advantageously be used in a cleaning-in-place (C.l.P.) system for cleaning of process equipment of any kind.

The antimicrobial polypeptides found by the present invention may additionally be used for cleaning surfaces and cooking utensils in food processing plants and in any area in which food is prepared or served such as at hospitals, nursing homes, restaurants, especially fast food restaurants, delicatessens and the like.

In general it is contemplated that the antimicrobial polypeptides found by the present invention may be useful for cleaning, disinfecting or inhibiting microbial growth on any hard surface.

Examples of surfaces, which may advantageously be contacted with the antimicrobial polypeptides of the invention are surfaces of process equipment used e.g. dairies, chemical or pharmaceutical process plants, water sanitation systems, oil processing plants, paper pulp processing plants, water treatment plants, and cooling towers. The antimicrobial polypeptides found by the present invention should be used in an amount, which is effective for cleaning, disinfecting or inhibiting microbial growth on the surface in question.

### INDUSTRIAL APPLICATIONS

Typically, antimicrobial polypeptides are useful at any locus subject to contamination by bacteria, fungi, yeast or algae, including places such as aqueous systems, e.g. water cooling systems, laundry rinse water, oil systems such as cutting oils, lubricants, oil fields and the like, where microorganisms need to be killed or where their growth needs to be controlled. However, the antimicrobial polypeptides found by the present invention may also be used in all applications for which known antimicrobial compositions are useful, such as protection of wood, latex, adhesive, glue, paper, cardboard, textile, leather, plastics, caulking, and feed. It may also be used as a preservation agent or a disinfection agent in water based paints. The antimicrobial polypeptides found by the present invention may also be useful for microbial control of water lines, and for disinfection of water, in particular for disinfection of industrial water.

Another example is use of an AMP of the present invention for preservation of enzyme formulations.

### EXAMPLES

Materials

Host Cells

E. coli origami: E.coli origami cells (Novagen) are a K-12 derivative strain harbouring mutations in both the thioredoxin reductase (trxB) and gluthathione reductase (gor) genes allowing disulfide bond formation in the cytoplasm.

E.coli TOP10: E.coli cells in which disulfide bond formation is not possible from Invitrogen

Antimicrobial Peptides

Plectasin is an antimicrobial peptide derived from Pseudoplectania nigrella (SEQ ID NO:2 in PA 2001 01732).

AFP is an antifungal protein derived from Aspergillus giganteus

Novispirin G10 is an alpha-helical antimicrobial polypeptide which does not contain any disulfide bonds. Novispirin G10 (WO 02/00839 SEQ ID No. 17) is obtained by rational design based on homology to SMAP-29, an ovine cathelicidin peptide.

AMP/Plasmid Constructs

pDR18-Plectasin: The AMP Plectasin was cloned into a version of the pBAD/glll A plasmid made in-house which does not comprise the gill secretion signal, whereby Plectasin is expressed in the cytoplasm of the host cell.

The promoter of both of the above pBAD/glll A plasmids is regulated by arabinose, i.e. expression of a polynucleotide sequence inserted into the plasmid is induced by the presence of arabinose.

pHHA: Is the same plasmid as pDR18-Plectasin but without the nucleotide sequence encoding Plectasin (i.e. it is the control vector for expression of Plectasin).

pHHA-Cm: Is the same plasmid as pHHA but where the nucleotide sequence encoding ampicillin resistance has been exchanged with a nucleotide sequence encoding chloramphenicol resistance.

pDR18-Cm-Plectasin: The pHHA-Cm plasmid into which the nucleotide sequence encoding Plectasin has been cloned.

pDR54, pDR55, pDR56, pDR57 and pDR58: Different variants of Plectasin created by site-directed mutagenesis and cloned into a version of the pBAD/gIII A plasmid made in-house which does not comprise the gill secretion signal, whereby the variants are expressed in the cytoplasm of the host cell.

pHH: The pBad/gIII plasmid comprising the gill secretion signal which directs expression of polypeptides into the periplasm.

pDRS5-Novispirin G10: The pHH plasmid into which the nucleotide sequence encoding Novispirin G10 was cloned.

pHHA900-AFP: Construct containing the AFP encoding sequence in the pBAD/glll plasmid where the promoter is regulated by arabinose and the expression of AFP is directed to the cytoplasm.

Indicator Cells

Bacillus subtilis

Bacillus subtilis 1315-1: a Bacillus subtilis strain which is resistant to chloramphenicol Fusarium longypes

Staphylococcus carnosus

E.coli TOP10 cell (see above for further description).

Culture Media

The composition of the RM media is described in the protocol from Invitrogen relating to the use of the pBAD/glll A, B and C plasmids (V450-01).

Methods

Radial Diffusion Assay

The protocol describing the radial diffusion assay method used in below examples is described in the patent WO 03/044049A1 based on the protocol published in (Lehrer et al., (1991) Ultrasensitive assays for endogenous antimicrobial polypeptides J Immunol Methods 137: 167-173) with some modifications. Briefly, Target bacteria (5x10⁶ colony forming units (CFU) were added to 50 ml of underlay agarose (1% low electro-endosmosis agarose, 0.03% Trypticase soy broth, 10 mM sodium phosphate, pH 7.4, 37 degrees C.). Suspension was solidified on a Nunc omnitray plate containing a nunc-ImmunoTSP PS rack inside to obtain 96 holes in the agarose media. Samples were added to the holes and incubated at 37 degrees C. for 3 hours. A 25 ml overlay of LB media with agar was poured on top and the plate was incubated overnight (LB media, 7.5% Agar). Antimicrobial activity was detected as bacterial clearing zones around the wells. Living cells were counterstained by adding 6 ml, 6 mM MTT (3-(4,5-Dimethylthiazol-2-yl)-2,5-diphenyltetrazolium bromide Thiazolyl blue). All standard protocols have been described elsewhere (Sambrook, Fritsch, and Maniatis, 1989).

Lysis of Cells With Hot Acid

Cells which were cultured in 96-well microtiter plates were lysed by hot acid by adding 100 microlitre of sodium phosphate buffer 1 M pH 2.3 to the wells. The final pH was approximately 3. The plates were incubated overnight at 80 degrees C. in a low condensation incubator with shaking. Next day, 75 microlitre of sodium phosphate buffer 0.5 M pH 13 was added to the wells. The final pH was approximately 7.

### Example 1

Growth Inhibition of Bacillus subtilis by the Antimicrobial Peptide Plectasin

E. coli origami cells were transformed with pDR18-Plectasin and cultured overnight at 37° C. on LB-agar plates containing tetracycline (tet), kanamycin (kan) and ampicillin (amp) to select for viable origami cells comprising the pDR18-Plectasin construct. Three viable origami colonies were selected and transferred to a cellulose acetate filter (Schleider & Schull) placed on top of an agar plate without any antibiotics. The filter-agar plates were incubated overnight at 37° C. before transferring the filters to a new agar plate containing 0.1% arabinose to induce expression of Plectasin. The filter-agar, 0.1% arabinose plates were incubated overnight at 37 degrees C. One of the plates were treated with vapours of chloroform, which lyses cells, by pouring chloroform on a filter and then placing the agar-filter plate on top of this upside-down for 10 minutes. Afterwards the filter-agar plate is remove and left open for 15 minutes to evaporate the remaining chloroform before transferring the filter to a new agar-filter, 0.1% arabinose plate. After this a top-layer of agar comprising approximately 4×10⁶ CFU Bacillus subtilis were poured on top of both the chloroform treated filter-agar plate and the non-treated plate. The plates with top-layer were then incubated overnight at 37 degrees C. The next day clearing zones in the top-layer (Bacillus subtilis cells) were detected by staining with a 0.2 mM MTT-solution, which stains viable cells blue. Thus the presence of clearing zones (no blue cells) surrounding the origami colonies indicates that the proliferation of the Bacillus subtilis cells near the origami cells is inhibited and thereby that the polynucleotide expressed by said host cells has an antimicrobial activity towards said indicator cells.

The results are shown in table 1 as whether or not a clearing zone is present in the top-layer over each of 6 colonies of origami cells transformed with the pDR18-Plectasin construct. A clearing zone is defined as the absence of or presence of only a few blue cells in the top-layer over each of the origami colonies, which indicates that the Bacillus subtilis cells, which are more or less uniformly distributed throughout the top-layer, above the origami colonies are dead.

TABLE 1

| | Plate treated | Plate not treated with |
|---|---|---|
| | with chloroform | chloroform |
| | Clearing zone | |
| AMP/plasmid construct | +/− | |
| pDR18-Plectasin no. 1 | + | + |
| pDR18-Plectasin no. 2 | + | + |
| pDR18-Plectasin no. 3 | + | + |

The above results show that expression of Plectasin by the origami cells is able to act on other cells (in this case Bacillus subtilis) than the host cells it is expressed by and kill those cells. Furthermore, it also shows that this effect is independent on whether or the host cells are lysed with chloroform.

### Example 2

The Effect of Plectasin on the Host Cells (E.coli Origami)

E. coli origami cells were transformed with pDR18-Plectasin and cultured overnight at 37 degrees C on LB-agar plates containing tet, kan and amp to select for viable origami cells comprising the pDR1 8-Plectasin construct. Four viable origami colonies were selected and two were streaked out on agar plates comprising tet, kan and amp while the other two were streaked out on agar plates comprising tet, kan, amp and 0.1% arabinose to induce expression of Plectasin from the pDR18-Plectasin construct. The plates were incubated overnight at 37 degrees C.

Results:

On the agar plates without arabinose a line of origami cells was present clearly indicating that the origami cells had grown overnight.

On the agar plates comprising arabinose only very few origami cells were present indicating that the presence of arabinose had inhibited the proliferation of said cells. Thus that the proliferation of the origami cells was almost completely inhibited by the presence of the AMP Plectasin as arabinose induce expression of Plectasin.

### Example 3

The Effect of Plectasin on Host Cells Not Capable of Forming Disulfide Bridges

TOP10 cells were transformed with pDR18-Plectasin and cultured overnight at 37 degrees C. on LB-agar plates containing amp to select for viable TOP10 cells comprising the pDR18-Plectasin construct. Four viable TOP10 colonies were selected and two were streaked out on agar plates comprising amp while the other two were streaked out on agar plates comprising amp and 0.1% arabinose to induce expression of Plectasin from the pDR18-Plectasin construct. The plates were incubated overnight at 37 degrees C.

Results:

A line of TOP10 cells was present at both the plates comprising arabinose and those without arabinose. Thus induction of Plectasin by arabinose did not inhibit proliferation of the TOP10 cells indicating that formation of disulfide bond(s) is important for the antimicrobial activity of Plectasin as expression of Plectasin in origami cells, in which disulfide bond formation is possible, inhibited proliferation of these cells (see example 2).

### Example 4

Growth Inhibition of Bacillus subtilis by Plectasin Diffused into Solid Media.

The method used in this example is similar to the method described in example 1 with the exception that in the present example the acetate filter containing the origami cells is removed from the arabinose plate before it is overlaid with the indicator strain.

E. coli origami cells were transformed independently with pHHA or pDR18-Plectasin plasmid and cultured overnight at 37 degrees C. on LB-agar plates containing tetracycline (tet), kanamycin (kan) and ampicillin (amp) to select for viable origami cells comprising the plasmid. Six viable origami colonies were selected containing each of the constructs and transferred to a cellulose acetate filter placed on top of an agar plate without any antibiotics. The filter-agar plates were incubated two nights at 37 degrees C. Next, the filters were transferred to a new agar plate containing 0.1% arabinose to induce expression of the peptide. The filter-agar, 0.1% arabinose plates were incubated overnight at 37 degrees C. Next day, the acetate filter was removed from the plates and a top-layer of agar comprising approximately 3×10⁵ CFU Bacillus subtiliswere poured on top of the plate. The plates with top-layer were then incubated overnight at 37 degrees C. The next day clearing zones in the top-layer (Bacillus subtilis cells) were detected by staining with a 0.2 mM MTT-solution, which stains viable cells blue.

The results are shown in table 2 as the presence (“+”) or absence (“−”) of a clearing zone in the top-layer over the place where each of the 6 colonies origami cells transformed with the pHHA/pDR18-Plectasin construct had been growing before removing the acetate filter. A clearing zone is defined as the absence of or presence of only a few blue cells in the top-layer, which indicates that the Bacillus subtilis cells, which are more or less uniformly distributed throughout the top-layer, are dead.

TABLE 2

| AMP/plasmid construct | Clearing zone (−/+) |
|---|---|
| pHHA no. 1 | − |
| pHHA no. 2 | − |
| pHHA no. 3 | − |
| pDR18-Plectasin no. 1 | + |
| pDR18-Plectasin no. 2 | + |
| pDR18-Plectasin no. 3 | + |

Compared to the results of example 1 this results indicate that the Plectasin is capable of diffusing through the acetate filter into the agar after it is produced by the E.coli origami cells.

### Example 5

Growth Inhibition of Bacillus subtilis by Plectasin Diffused into Solid Media without Sub-Culturing the Host Cells Expressing Plectasin

The method used in this example is similar to the method described in example 4 with the exception that only ampicillin was used as a selection marker after transformation of the E.coli origami cells and beta-lactamase was added to the top-layer of agar comprising Bacillus subtilis. Beta-lactamase is capable of degrading ampicillin and thereby allowing the Bacillus subtilis to grow.

In brief, E.coli origami cells were transformed independently with pHHA or pDR18-Plectasin plasmid and cultured for 2 nights at 37 degrees C. on a LB-agar plate comprising ampicillin (amp) to select for viable E.coli origami cells comprising the plasmid. Thereafter, an acetate filter was placed on top the agar plate comprising the transformed cells and the acetate filter was then stripped off and transferred to a another agar plate comprising 0.1% arabinose and ampicillin. The plate comprising the acetate filter was incubated overnight at 37 degrees C. Next day, the acetate filter was removed from the plate and a top-layer of agar comprising 0,25 Units of beta-lactamase and approximately 3×10⁵ CFU Bacillus subtilis was poured on top of the plate. The plate with top-layer was incubated overnight at 37 degrees C. The next day, clearing zones in the top-layer (Bacillus subtilis cells) were detected by staining with a 0.2 mM MTT-solutions, which stains viable cells blue.

The results were similar to the results of example 4, i.e. there were no clearing zones present in the top-layer comprising Bacillus subtilis in the areas corresponding to where the E.coli origami cells expressing the control plasmid pHHA had been growing, while clearing zones were present in the areas corresponding to where the E.coli origami cells expressing the Plectasin-expressing plasmid pDR-Plectasin had been growing.

The present example provides a method where it is not necessary to sub-culture the host cells.

### Example 6

Growth Inhibition of Bacillus subtilis by Plectasin Variants Diffused into Solid Media without Sub-Culturing the Host Cells Expressing the Variants

The method used in this example is similar to the one described in example 5. In brief, E.coli origami cells were transformed independently with pHHA (empty control vector), pDR18-Plectasin (wild type Plectasin) and five constructs encoding for different Plectasin variants (pDR54, pDR55, pDR56, pDR57 and pDR58) and plated on LB-agar plates with ampicillin and incubated overnight at 37 degrees C. to select for viable E.coli origami cells comprising the construct.

Four viable E.coli origami colonies from each construct were picked and transferred to a cellulose acetate filter placed on top of an agar plate with ampicillin. The filter-agar plate was incubated two nights at 37 degrees C. before transferring the filter to a new agar plate comprising ampicillin and 0.1% arabinose to induce expression of Plectasin/Plectasin variants.

The plate was incubated overnight at 37 degrees C. Next day, the acetate filter was removed from the plate and a top-layer of agar comprising 0.25 Units of beta-lactamase and approximately 3×10⁵ CFU Bacillus subtilis were poured on top of the plate. The plate with top-layer was then incubated overnight at 37 degrees C. before staining with 0.2 mM MTT-solution to detect clearing zones in the top-layer.

Results:

Clearing zones were present in the top-layer comprising Bacillus subtilis corresponding to the areas where E.coli origami cells comprising pDR-Plectasin (wild-type Plectasin) or pDR54, pDR55, pDR56, pDR57 and pDR58 (Plectasin variants) had been growing before removing the acetate filter, while no clearing zone was present in the area corresponding to where the E.coli origami cells comprising pHHA (empty control vector) had been growing. Furthermore, the clearing zones were larger and more defined at the areas where the E.coli origami cells expressing the Plectasin variants had been growing than were the E.coli origami cells expressing wild-type Plectasin had been growing. This indicates that the Plectasin variants have a higher antimicrobial activity towards Bacillus subtilis than wild-type Plectasin has.

### Example 7

Growth Inhibition of a Chloramphenicol-Resistant Strain of Bacillus subtilis by Plectasin Diffused into Solid Media

The method used in this example is similar to the method used in example 4 with the exception that chloramphenicol was used as a selection marker and a chloramphenicol-resistant strain of Bacillus subtilis (1315-1) was used as indicator strain.

In brief, E. coli origami cells were transformed independently with pHHA-Cm and pDR18-Cm-Plectasin constructs and cultured overnight at 37 degrees C on LB-agar plates comprising chloramphenicol to select for viable E.coli origami cells comprising the construct. Six viable origami colonies from each construct were picked and transferred to a cellulose acetate filter placed on top of an agar plate with chloramphenicol. The filter-agar plate was incubated for two nights at 37 degrees C before transferring the filters to an agar plate comprising chloramphenicol and 0.1% arabinose to induce expression of Plectasin. The plates were incubated overnight at 37 degrees C. Next day, the acetate filter was removed from the plate and a top-layer of agar comprising approximately 3×10⁵ CFU Bacillus subtilis (1315-1) resistant to chloramphenicol were poured on top of the plate. The plates with top-layer were then incubated overnight at 37 degrees C. before staining with 0.2 mM MTT-solution to detect clearing zones in the top-layer comprising the Bacillus subtilis cells (1315-1).

Results:

The results are shown in table 3 as the presence “+” or absence “−” of a clearing zone in the top-layer corresponding to the area where E.coli origami cells comprising either the pHHA-Cm or pDR18-Cm-Plectasin constructs had been growing. A clearing zone is defined as the absence of or presence of only a few blue cells in the top-layer, which indicates that the Bacillus subtilis cells (1315-1), which are more or less uniformly distributed throughout the top-layer, are dead.

TABLE 3

| AMP/plasmid construct | Clearing zone (−/+) |
|---|---|
| pHHA-Cm no. 1 | − |
| pHHA-Cm no. 2 | − |
| pHHA-Cm no. 3 | − |
| pHHA-Cm no. 4 | − |
| pHHA-Cm no. 5 | − |
| pHHA-Cm no. 6 | − |
| pDR18-Cm-Plectasin no. 1 | + |
| pDR18-Cm-Plectasin no. 2 | + |
| pDR18-Cm-Plectasin no. 3 | + |
| pDR18-Cm-Plectasin no. 4 | + |
| pDR18-Cm-Plectasin no. 5 | + |
| pDR18-Cm-Plectasin no. 6 | + |

Compared to the previous examples these results indicate that different selection markers may be used depending on whether or not the indicator strain is resistant or not to the selection marker.

### Example 8

Growth Inhibition of Bacillus subtilis by Plectasin Diffused into Solid Media without Using Cellulose Acetate Filters

Briefly, E. coli origami cells were transformed independently with pHHA (control empty vector) and pDR18-Plectasin (wild type Plectasin) and the transformed cells were plated on LB-agar plates with ampicillin and incubated overnight at 37 degrees C. to select for viable E.coli origami cells comprising the plasmid constructs.

For each plasmid construct three viable E.coli origami colonies were picked and transferred to an LB-agar plate and incubated for three days at 37 degrees C. Next, the plate was overlaid with a top layer of agar comprising 0.2% arabinose and incubated overnight at 37 degrees C. to induce expression of Plectasin. Next day, the plate was overlaid with a top-layer of agar comprising approximately 3×10⁵ CFU Bacillus subtilis. The plate was then incubated overnight at 37 degrees C. before staining with 0.2 mM MTT-solution to detect clearing zones in the top-layer comprising the Bacillus subtilis cells

Results:

The results are shown in table 4 as the presence “+” or absence “−” of a clearing zone in the top-layer corresponding to the area where E.coli origami cells comprising either the pHHA or pDR18-Plectasin plasmid constructs had been growing. A clearing zone is defined as the absence of or presence of only a few blue cells in the top-layer, which indicates that the Bacillus subtilis cells, which are more or less uniformly distributed throughout the top-layer, are dead.

TABLE 4

| AMP/plasmid construct | Clearing zone (−/+) |
|---|---|
| pHHA no. 1 | − |
| pHHA no. 2 | − |
| pHHA no. 3 | − |
| pDR18-Plectasin no. 1 | + |
| pDR18-Plectasin no. 2 | + |
| pDR18-Plectasin no. 3 | + |

In comparison to the previous examples the present results show that the same results are obtained as previous (Plectasin expressed by E.coli origami cells is able to inhibit the growth of Bacillus subtilis) even when an acetate filter is not used.

### Example 9

High-Through-Put Screening of Plectasin Variants for Growth Inhibition of Staphylococcus carnosus in a Liquid Microtiter Format.

The nucleotide sequence encoding Plectasin was randomly mutagenized by Error-Prone PCR to create a library of nucleotide sequences encoding Plectasin variants. The library was cloned into the pHHA vector and transformed into E.coli origami cells. Transformed colonies were grown overnight at 37 degrees C. on LB-agar plates containing ampicillin and 0.2% glucose to select for viable origami cells comprising the plasmid. Glucose was added to reduce the basal expression level of the pBAD promoter. Colonies were picked using a colony picker and inoculated in microtitter plates containing 200 microlitre of liquid media, either LB or TB, and ampicillin. Cultures were grown overnight at 37 degrees C. in a low condensation incubator (Küjhner) with shaking. Next day, 15 microlitre of the overnight cultures were transferred to new microtitter plates containing fresh media, either LB or TB, with ampicillin. Plates were shaken for 4 hours at 37 degrees C. in a low condensation incubator before 15 microlitre of 1% arabinose was added to the cultures to induce expression of the Plectasin-variants. Plates were incubated overnight at 37 degrees C. in a low condensation incubator with shaking. Next day, a hot acid hydrolysis of the cultures was performed and 25 microlitre of the hydrolyzed cultures was analyzed on a radial diffusion assay to test for antimicrobial activity against Staphylococcus carnosus.

Results are shown in table 5 (see below) as whether or not a clearing zone is present in the agar media (“−” or “+”) or if the clearing zones are particular large (“++”).

TABLE 5

| | 1 | 2 | 3 | 4 | 5 | 6 | 7 | 8 | 9 | 10 | 11 | 12 |
|---|---|---|---|---|---|---|---|---|---|---|---|---|
| A | − | + | + | ++ | ++ | − | − | − | − | − | − | − |
| B | − | − | − | − | − | ++ | − | − | − | + | − | − |
| C | − | − | − | − | − | − | − | − | − | − | − | + |
| D | − | + | − | − | − | − | − | − | − | + | − | − |
| E | − | + | − | − | − | − | ++ | − | + | − | − | − |
| F | − | − | − | − | − | − | − | + | − | − | + | − |
| G | ++ | − | + | − | − | − | − | − | − | − | + | − |
| H | − | − | − | − | − | − | − | − | ++ | − | − | − |

Results presented in table 5 showed that very week clearing zones were detected on the radial diffusion plates from the lyzed cultures expressing Plectasin wild type (A2 and A3) or from some of the Plectasin variants (B10, C12, D2, D10, E2, E9, F8, F11, G3, G11). No clearing zones were observed from the control plasmid (Al). In contrast, larger clearing zones were detected on some of the lyzed cultures expressing different Plectasin variants (A4, A5, B6, E7, G1 and H9). These results indicate that this assay can discriminate between E.coli cells expressing peptide variants with different antimicrobial activity and it can be used to find peptides with improved antimicrobial activity.

### Example 10

Test of the Antimicrobial Activity of Different Plectasin Variants on Staphylococcus camosus in a Liquid Assay using Chloroform to Lyse the Cells.

E.coli origami cells were transformed with different constructs (plasmids): pHHA (control empty vector), pDR18-Plectasin (wild type Plectasin) and pDR54, pDR55, pDR56, pDR57 and pDR58 (Plectasin variants). Transformed cells were plated on LB-agar plates with ampicillin and incubated overnight at 37 degrees C. to select for viable origami cells containing the plasmids.

For each construct two viable origami colonies were picked and inoculated into 5ml LB with ampicillin. The tubes were incubated overnight at 37 degrees C. with shaking. Next day, 500 microlitre of the inoculated cultures were transferred to tubes containing 5 ml fresh LB with ampicillin. Two tubes were prepared from each overnight culture. The cultures were then grown at 37 degrees C. with shaking for approximately 4 hours until the OD₆₀₀ was approximately 1.5. For each construct 50 microlitre of 10% arabinose was then added to one of the tubes to induce polypeptide synthesis. The tubes were incubated overnight at 37 degrees C. with shaking. Next day, lysis of the cultures was performed using chloroform in the following way: 150microlitre of the overnight cultures were transferred to 1.5 ml tubes and 50 microlitre of chloroform was added to each tube. The tubes were inverted several times to mix the samples and incubated for 30 minutes at room temperature. Next, 100 microlitre of the upper phase of each tube was transferred to a new tube. Finally, an aliquot of 15 microlitre of the lyzed cultures was analyzed on a radial diffusion assay to test for antimicrobial activity against Staphylococcus carnosus.

Results are shown in table 6 (see below) as whether or not a clearing zone is present in the agar media (“−” or “+”) or if the clearing zones are larger (“++”). Columns 1, 3, 5, 7, 9 and 11 correspond to samples not treated with arabinose. Columns 2, 4, 6, 8, 10 and 12 correspond to samples treated with arabinose. Samples A1-A4 correspond to control vector pHHA, A5-8 correspond to pDR18-plectasin, A9-12 correspond to pDR54, B1-4 correspond to pDR55, B5-8 correspond to pDR56, and B9-12 correspond to pDR57 and C1-2 correspond to pDR58.

TABLE 6

| | 1 | 2 | 3 | 4 | 5 | 6 | 7 | 8 | 9 | 10 | 11 | 12 |
|---|---|---|---|---|---|---|---|---|---|---|---|---|
| A | − | − | − | − | − | + | − | + | − | + | − | + |
| B | − | ++ | − | ++ | − | ++ | − | ++ | − | ++ | − | ++ |
| C | − | ++ | | | | | | | | | | |

Results in table 6 show that cells treated with arabinose expressing wild type Plectasin and the five variants gave clearing zones in the radial assay. No clearing zones were observed from the cells comprising the control plasmid or from the cells not induced with arabinose. Additionally, four Plectasin variants, pDR55, pDR56, pDR57 and pDR58 showed bigger clearing zones than wild type, indicating that the antimicrobial activity of these variants was higher than the wild type. However, the size of the clearing zone from the variant pDR54 was similar to the one from cells expressing the wild type Plectasin, suggesting that the antimicrobial activity of this peptide was similar to the wild-type Plectasin. These results indicate that this assay can discriminate between E.coli cells expressing peptide variants with different antimicrobial activity.

### Example 11

Test of the Antimicrobial Activity of Different Plectasin Variants on Staphylococcus carnosus in a Liquid Assay using Hot Acid to Lyse the Cells.

This example was performed similar to example 9, with the exception that instead of testing the antimicrobial activity of a library of Plectasin variants on Staphylococcus carnosus the antimicrobial activity of wild-type Plectasin was compared with that of the Plectasin variants encoded by pDR54, pDR55, pDR56, pDR57 and pDR58 was tested. In brief, E.coli origami cells were transformed with pHHA (control empty vector), pDR18-Plectasin (wild type plectasin) and pDR54, pDR55, pDR56, pDR57 and pDR58 (Plectasin variants). Three viable transformed origami colonies from each construct were picked and cultured first in the absence of arabinose (polypeptide inducer) and subsequently in the presence of arabinose (as described in example 9). The cells were then lysed by hot acid and 25 microlitre of each cell-culture was analyses on a radial diffusion assay to test for antimicrobial activity against Staphylococcus carnosus.

Results are shown in table 7 (see below) as whether or not a clearing zone is present in the agar media (“−” or “+”) or if the clearing zones are larger (“++”). Samples Al-A3 correspond to control vector pHHA, A4-6 correspond to pDR18-plectasin, A7-9 correspond to pDR54, A10-12 correspond to pDR55, B1-3 correspond to pDR56, and B4-6 correspond to pDR57, B7-9 correspond to pDR58, and B10-12 correspond to Blank.

TABLE 7

| | 1 | 2 | 3 | 4 | 5 | 6 | 7 | 8 | 9 | 10 | 11 | 12 |
|---|---|---|---|---|---|---|---|---|---|---|---|---|
| A | − | − | − | − | − | − | + | + | + | + | + | + |
| B | + | + | + | ++ | ++ | ++ | ++ | ++ | ++ | − | − | − |

Results in table 7 showed that no clearing zones were obtained from the hot acid hydrolyzed cultures corresponding to the control pHHA plasmid, or wild type Plectasin (pDR18) or from liquid media without E.coli cells (Blank). In contrast, the hot acid hydrolyzed cultures corresponding to the five Plectasin variants, pDR54, pDR55, pDR56, pDR57 and pDR58, showed clearing zones, indicating that the antimicrobial activity of these variants was higher than the wild type. Additionally, the clearing zones obtained from pDR57 and pDR58 were bigger than the ones obtained from the other three variants, suggesting that their antimicrobial activity was probably higher than for the other variants.

### Example 12

Growth Inhibition of E.coli TOP10 by Novispirin G10 in a Liquid Assay using Hot Acid to Lyse the Cells

This example was performed similar to examples 9 and 11 with the following exceptions:

In the present example the antimicrobial activity of Novispirin G10 was tested towards E.coli TOP10 cells.

Novispirin G10 was expressed in E.coli TOP10 cells which do not carry the trxB and gor mutations as E.coli origami cells, since the activity of the antimicrobial polypeptide does not require the formation of disulfide bonds in the molecule.

The expression of Novispirin G10 was directed to the periplasm, instead of the cytoplasm, which was the case of the other antimicrobial polypeptides tested in the other examples.

Last, in this experiment, E.coli TOP10 was used as indicator cells. Briefly, E. coli Top10 cells were transformed with pHH (control empty vector), and pDRS5-Novispirin G10. Three viable transformed E. coli TOP10 colonies from each construct were picked and cultured first in the absence of arabinose and subsequently in the presence of arabinose (as described in example 9). The cells were then lysed with hot acid and 25 microlitre of each cell-culture was analysed on a radial diffusion assay to test for antimicrobial activity of Novispirin G10 against E. coli TOP10.

Results are shown in table 8 as whether or not a clearing zone is present in the agar media (“−” or “+”). Samples A1-3 corresponds to the control vector pHH and samples A4-A6 corresponds to the pDRS5-Novispirin G10.

TABLE 8

| | 1 | 2 | 3 | 4 | 5 | 6 |
|---|---|---|---|---|---|---|
| A | − | − | − | + | + | + |

Results in table 8 showed that no clearing zones were obtained from the cultures transformed with the control vector pHH. In contrast, the cultures transformed with Novispirin G10, showed clearing zones. These results indicate that E.coli TOP10 cells are sensitive to the antimicrobial polypeptide Novispirin G10.

### Example 13

Growth Inhibition of E. coli TOP10 Upon Expression of Novispirin G10

In order to evaluate whether E. coli TOP10 as host cells are sensitive to Novispirin G10, the following experiment was conducted in liquid media as disclosed in example 1 of patent application WO 00/73433 (under “Growth inhibition of E.coli upon expression of various AMP's”) with some modifications. Briefly, fresh overnight cultures of cells containing either the pHH (control) or pDRS5-Novispirin G10 plasmid were diluted 300-fold into 150 micro liter of RM media or RM media containing 0.1% arabinose in a microtiter plate and incubated at 37 degrees C. with vigorous shaking. The growth curve was monitored by measuring OD450 at regular intervals using an ELISA reader. The percentage of growth inhibition was calculated by taking the end point OD measurement of each sample divided by the end point OD measurement obtained from cells containing the control vector and multiplied by 100. The formula is as follows: (1-(sample OD-empty well OD/control vector OD-empty well OD)), where the empty well value correspond to a well where no cells had been growing (Blank).

Results are presented in table 9 and show that Novispirin G10 inhibited 90% cell growth when expression of Novispirin G10 was directed to the periplasm, in contrast to the control vector that only inhibited 18% (see below).

TABLE 9

| Plasmid | % inhibition |
|---|---|
| pHH | 18% |
| pDRS5-Novispirin G10 | 90% |

These results indicate that the host cells, E. coli TOP10 are sensitive to Novispirin G10 when they are expressing it in the periplasm.

### Example 14

Growth Inhibition of Fusarium longpyes by AFP

E. coli origami cells were transformed with pHHA900-AFP and pHHA, respectively and cultured overnight at 37 degrees C. on LB-agar plates containing tetracycline (tet), kanamycin (kan) and ampicillin (amp) to select for viable origami cells comprising said constructs. Two viable origami colonies containing each of the constructs were selected and used to inoculate 3 other LB-agar plates with tet, kan and amp before culturing said plates for 3 days at 37 degrees C. A cellulose acetate filter (Schleider & Schull) was placed on top of each of the plates and the colonies were striped off with the filters. The filters were then place on new LB-agar plates containing 0.1% arabinose with the colonies facing up to induce expression of the AFP. The filter-agar, 0.1% arabinose plates were incubated overnight at 37 degrees C. before treating them with chloroform as described in example 1. After this a top-layer of 6 ml agar comprising approximately 103 spores/ml Fusarium longypes were poured on top of the chloroform treated filter-agar plate. The plates with top-layer were then incubated for 3 days at room temperature. The pink color of the Fusarium longypes mycelium facilitated the detection of the clearing zones in the top layer. The results are shown in table 10 as whether or not a clearing zone is present in the top-layer over each of 2 colonies of origami cells transformed with the pHHA900-AFP and pHHA construct, respectively. A clearing zone is defined as the absence of or presence of only a bit of mycelium in the top-layer over each of the origami colonies, which indicates that the Fusarium longypes mycelium, which is more or less uniformly distributed throughout the top-layer, above the origami colonies has not grown.

TABLE 10

| | Plate treated with chloroform |
|---|---|
| AMP/plasmid construct | Clearing zone +/− |
| pHHA900-APF no. 1 | + |
| pHHA900-AFP no. 2 | + |
| pHHA no. 1 | − |
| pHHA no. 2 | − |

The results shown in table 10 shows that the indicator cells Fusarium longypes present on top of the origami cells transformed with the construct comprising AFP are dead, while origami cells which were transformed with the control vector had no effect on the Fusarium longypes indicator cells. Thus this indicates that expression of AFP by the origami cells is capable of killing or inhibiting the Fusarium longypes cells.

### Example 15

Growth Inhibition of Fusarium longypes by AFP Diffused into Solid Media

The method used in this example is similar to the method described in example 14 with the difference that in this example the acetate filter containing the E.coli origami cells is removed from the arabinose plate before it is overlaid with the fungus Fusarium longypes which is used as an indicator strain.

Additionally, this example is very similar to example 4, with the exception that in the present example AFP is the antimicrobial polypeptide (instead of Plectasin in example 4) and Fusarium longypes is the indicator cells (instead of Bacillus subtilis in example 4). As described in example 14, E. coli origami cells were transformed with pHHA900-AFP and pHHA, respectively and cultured overnight at 37 degrees C on LB-agar plates containing tetracycline (tet), kanamycin (kan) and ampicillin (amp) to select for viable E.coli origami cells comprising said constructs. Two viable E.coli origami colonies containing each of the constructs were selected and used to inoculate a LB-agar plate with tet, kan and amp before culturing said plate for 3 days at 37 degrees C. A cellulose acetate filter was placed on top of the plate and the colonies were striped off with the filter. The filter was then placed on a new LB-agar plate containing 0.1% arabinose with the colonies facing up to induce expression of the AFP. The filter-agar, 0.1% arabinose plate was incubated overnight at 37 degrees C. Next day, in this example, however, instead of treating the filter with chloroform as described in example 14, the acetate filter was removed from the plate as described in example 4. After this, a top-layer of 6 ml agar comprising approximately 103 spores/ml Fusarium longypes was poured on top of the plate. The plate with top-layer was then incubated for 3 days at room temperature. The pink colour of the Fusarium longypes mycelium facilitated detection of clearing zones in the top layer.

The results are shown in table 11 as whether or not a clearing zone is present in the top-layer over the place where each of the 2 E.coli origami colonies transformed with either pHHA900-AFP or pHHA construct had been growing before removing the acetate filter. A clearing zone is defined as the absence of or presence of only a bit of mycelium in the top-layer, which indicates that the Fusarium longypes mycelium, which is more or less uniformly distributed throughout the top-layer, has not grown.

TABLE 11

| AMP/plasmid construct | Clearing zone +/− |
|---|---|
| pHHA900-APF no. 1 | + |
| pHHA900-APF no. 2 | + |
| pHHA no. 1 | − |
| pHHA no. 2 | − |

Compared to the results of example 14, the results in table 11 indicate that AFP is capable of diffusing through the acetate filter into the agar media after it is produced by the E. coli origami cells.

1. A method for screening a polynucleotide sequence encoding an antimicrobial polypeptide, said method comprising the steps of:
a) introducing the polynucleotide sequence in a host cell, wherein expression of said polynucleotide is under control of an inducible promoter and wherein said host cell is sensitive to the antimicrobial peptide encoded by the polynucleotide sequence
b) cultivating the host cells of in the absence of an inducer capable of inducing expression of the antimicrobial peptide
c) cultivating the host cells in the presence of an inducer capable of inducing expression of the antimicrobial peptide
d) cultivating the host cells in the presence of an indicator cell, wherein said indicator cell is sensitive to the antimicrobial polypeptide encoded by the polynucleotide sequence
e) selecting host cells capable of reducing the proliferation of indicator cells
f) recovering the polynucleotide sequence encoding an antimicrobial polypeptide from the host cells selected in step e).

2. A method according to claim 1, wherein step d) comprises cultivating the host cells in the presence of a library of indicator cells.

3. A method for screening a library of polynucleotide sequences encoding one or more antimicrobial polypeptide(s), comprising:
a) cultivating an indicator cell in the presence of the antimicrobial polypeptide(s), wherein the antimicrobial polypeptide(s) has/have been expressed by a host which is sensitive to said antimicrobial polypeptide(s), and wherein expression of each of the polynucleotide sequences in the library has been under control of an inducible promoter, and wherein the host cells comprising the library polynucleotide sequence has been cultivated in the absence of an inducer and subsequently in the presence of an inducer
b) selecting a host cell expressing an antimicrobial polypeptide which is capable of reducing the proliferation of the indicator cell.

4. A method according to claim 3, wherein a library of indicator cells are cultivated in the presence of the antimicrobial polypeptide(s).

5. A method for testing the antimicrobial activity of an antimicrobial polypeptide comprising step a) of claim 3.

6. A method according to claim 5, wherein a library of indicator cells are cultivated in the presence of the antimicrobial polypeptide.

7. A method according to claim 3, wherein the indicator cell is sensitive to one or more antimicrobial polypeptides.

8. A method according to claim 5, wherein the indicator cell is sensitive to the antimicrobial polypeptide.

9. A method according to claim 3, wherein said method further comprises the step of:
c) recovering the polynucleotide sequence encoding the antimicrobial polypeptide from the host cell.

10. A method according to claim 1, wherein the polynucleotide sequence comprises a library of polynucleotide sequences.

11. A method according to claim 1, wherein the antimicrobial polypeptide comprises disulfide bonds.

12. A method according to claim 1, wherein the antimicrobial polypeptide is expressed intracellularly in the periplasm or secreted by the host cell.

13. A method according to claim 12, wherein the antimicrobial polypeptide is expressed in the cytoplasm or in an intracellular compartment.

14. A method according to claim 1, wherein the host cell is a fungal cell.

15. A method according to claim 14, wherein the host cell is Aspergillus or yeast.

16. A method according to claim 1, wherein the host cell is a bacterial cell.

17. A method according to claim 16, wherein the host cell is a Gram-negative bacterial cell.

18. A method according to claim 17, wherein the host cell is E.coli.

19. A method according to claim 16, wherein the host cell is a Gram-positive bacterial cell.

20. A method according to claim 19, wherein the host cell is Bacillus subtilis.

21-25. (canceled)
